(19) United States
(12) Patent Application Publication
(10) Pub. No.: US 2024/0110150 A1
(43) Pub. Date: Apr. 4, 2024

(54) PHOTON UPCONVERSION BIOMATERIALS, MICELLE AND NANOPARTICLES FOR THREE-DIMENSIONAL (3D) OPTOGENETICS

(71) Applicant: THE TRUESTEES OF COLUMBIA UNIVERSITY IN THE CITY OF NEW YORK, New York, NY (US)

(72) Inventors: Luis M. CAMPOS, Pennington, NJ (US); Rinat MEIR (MEYER), Modiin (IL)

(21) Appl. No.: 18/205,040

(22) Filed: Jun. 2, 2023

Related U.S. Application Data
(63) Continuation of application No. PCT/US2021/061623, filed on Dec. 2, 2021.
(60) Provisional application No. 63/120,515, filed on Dec. 2, 2020.

(51) Int. Cl. C12N 5/00; C09K 11/02; C09K 11/06

(52) U.S. Cl. CPC C12N 5/0068; C09K 11/025; C09K 11/06; B82Y 15/00; C09K 2211/1011

(57) ABSTRACT

Exemplary embodiments of the present disclosure provides biomaterials, systems, and methods that utilize upconversion biomaterials to stimulate optogenetic cells in three-dimensional settings. Provided is a biomaterial including chromophores capable of converting low-energy light to high-energy light embedded in biocompatible materials. This technology enables more selective stimulation of optogenetic cells in three-dimensional scaffolds and has the potential to provide critical insights into cell function and disease.

### CROSS-REFERENCE TO RELATED APPLICATION(S)

This application is a continuation of International Patent Application No. PCT/US2021/061623, filed on Dec. 2, 2021 that published as WO 2022/120059 on Jun. 9, 2022, and also relates to and claims priority from U.S. Provisional Application No. 63/120,515, filed Dec. 2, 2020, the entire disclosures of which are incorporated herein by reference.

### STATEMENT REGARDING FEDERAL FUNDING

This invention was made with government support under 2039044, awarded by the National Science Foundation. The government has certain rights in this invention.

### FIELD OF THE DISCLOSURE

This disclosure relates to exemplary configurations to optically active, three-dimensional (3D) biomaterials (e.g., in form of hydrogels) to manipulate cellular behavior with low-energy light that is capable of penetrating deep tissues, and exemplary methods of monitoring and modulating cellular behaviors using the optically active, three-dimensional (3D) biomaterials with spatiotemporal control.

### BACKGROUND INFORMATION

The ability to manipulate optically-induced biological responses in three-dimensions (3D) has been dwarfed by the physical limitations of visible light penetration to trigger photochemical processes. However, many biological systems are relatively transparent to low-energy light, which does not provide sufficient energy to induce photochemistry in 3D. To overcome this challenge, we developed hydrogel biomaterials that are capable of converting red or near-infrared (NIR) light into blue light within the cell-laden 3D scaffolds. The upconverted light can then excite optically active proteins in cells to trigger a photochemical response. According to exemplary embodiments of the present disclosure, an exemplary upconversion gel biomaterial can be provided utilizing modular chromophores that can convert low energy red and NIR light to high energy visible blue light, by the photophysical process of triplet-triplet annihilation upconversion (TTA-UC). For example, cells were encapsulated within the biomaterial and found that the locally upconverted light can trigger an optogenetic response on-demand in 3D with excellent spatial resolution. This photoresponsive 3D cell culture platform is expected to open up new possibilities for dynamic control over cellular behavior with unprecedented specificity.

Optical stimulation of cells within 3D biomaterials has the great potential to precisely modulate cell function with unprecedented spatiotemporal control. For example, according to exemplary embodiments of the present disclosure, it was recognized that the use of light to stimulate cell excitation, inhibition and control signaling pathways could lead to unprecedented precision in the control of cellular behaviors in a 3D environment. A significant challenge in triggering optogenetic proteins in 3D with light arises from the fact that high energy visible photons are generally scattered and absorbed at the surface, and do not penetrate deeply into tissues, limiting progress in this area. While low-energy light (e.g., NIR) can deeply penetrate tissues, it does not possess the energy to activate most of the optogenetic proteins.

Accordingly there is a need to address and/or overcome at least some of the deficiencies and/or challenges described herein above.

### SUMMARY OF EXEMPLARY EMBODIMENTS

Thus, to overcome the above-described deficiencies and/or challenges, exemplary 3D biomaterials containing chromophores that are capable of photon upconversion by TTA-UC (also referred to as triplet fusion) according to exemplary embodiments of the present disclosure can be provided which is an important development in optoelectronic materials. This net process converts two low-energy photons into a single high-energy visible photon via triplet-triplet annihilation TTA-UC (also known as triplet fusion).

Exemplary embodiments of the present disclosure can implement two unique and innovative fundamental concepts that stem from physical organic chemistry, optoelectronic materials, biology, and tissue engineering: (i) adapting the concept of TTA-UC from various chromophores to facilitate deep penetration and provide high-energy visible light photons from within the biomaterials; (ii) controlling the activation of optogenetic cells using light, within 3D biomaterials; and (iii) selective optogenetic activation with single-cell resolution.

The biomaterials can be designed to be biocompatible (non-cytotoxic) and the intensity of the blue light generated by photon upconversion within the biomaterials can be sufficient to activate optogenetic proteins in, e.g., engineered multipotent stem cells, engineered pluripotent stem cells, and HeLa cells. This technology may enable to manipulate cellular behaviors and functions in 3D settings from a variety of cell lines.

Exemplary embodiments of the present disclosure can be utilized to meet the universal need for dynamic control over cellular behavior in 3D, using light as non-invasive stimulant to trigger optogenetic proteins with unprecedented specificity and precision.

According to an exemplary aspect of the present disclosure, provided is a biomaterial, such as a 3D biomaterial, comprising:

a biocompatible scaffold.

a cell, and

an emulsion droplet containing TTA-UC chromophores that emit visible light upon excitation with NIR or red light,

wherein the cell and the emulsion droplet are associated with the biocompatible scaffold.

The biocompatible scaffold may comprise or be composed of a hydrogel matrix. The hydrogel matrix may comprise a fibrinogen hydrogel. The hydrogel matrix may also comprise synthetic materials, such as polymer networks.

In the biomaterial, the cell may express a light activated protein, which may be fused to a molecule that modulates a cellular behavior. The light activated protein may be cryptochrome 2.

The TTA-UC chromophores used in the biomaterial may include a sensitizer chromophore and an annihilator chromophore. The chromophores may comprise at least one selected from the group consisting of diketopyrrolopyrrole, anthracene, tetracene, and other chromophores that undergo TTA-UC.

The emulsion, emulsion droplet or micelle in the biomaterial may contain a surfactant. Examples of the surfactant include Pluronic F127, Tween 80, and combinations thereof. Preferably, the surfactant comprises Pluronic F127. The emulsion may contain a micelle. The micelle may contain a natural oil.

According to another exemplary aspect of the present disclosure, provided is a micelle or emulsion droplet, comprising TTA-UC chromophores that emit a visible light upon excitation with near-infrared light, a surfactant and a natural oil.

According to another exemplary aspect of the present disclosure, provided is a nanoparticle, comprising the micelle according to the present disclosure and a layer containing silica disposed on at least a portion of a surface of the micelle.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying FIG.s showing illustrative embodiments of the present disclosure, which are as follows.

FIG. 1A) is an exemplary representation of the concept of upconversion optogenetics within hydrogel biomaterials by NIR light penetration and local emission of blue light.

FIG. 1B) is an illustration of exemplary components comprising the upconversion micrometer-sized droplets: surfactant, reductant oil (soybean oil), sensitizer, and annihilator according to an exemplary embodiment of the present disclosure.

FIG. 1C) is an exemplary photograph of the hydrogels in microwells without and with near-infrared (NIR) irradiation, where the irradiated gel emits blue light.

FIG. 1D) is an exemplary normalized absorption spectrum of the PtTPTNP sensitizer (red) and emission spectrum of the TIPS-An annihilator (blue).

FIG. 2A) is an exemplary graph of a photon upconversion in aerated conditions with triplet-triplet annihilation photon upconversion (TTA-UC) emulsions and relative biocompatibility also illustrating upconversion luminescence emission spectra of air saturated TTA-UC emulsions (TIPS-An:PdTPTBP) upon excitation with red light (637 nm) as measured after 1 min under constant irradiation using trimethylbenzene (TMB), soybean oil (SB) and D-limonene (DL), in the oil phase of the emulsion.

FIG. 2B) is an exemplary graph illustrating a stability of upconversion emulsions over 15 min under constant irradiation. Plot shows intensity at various time intervals, relative to the initial intensity of the system. Intensities were monitored at the emission maxima (450 nm).

FIG. 2C) is an exemplary graph of a biocompatibility of the TTA-UC emulsions with D-limonene and soybean oil. Cell viability was measured at 1 h incubation with emulsions-containing media at different volume percent. D-limonene was found to be toxic to the cells even at low volumes while soybean oil was found to be remarkably less cytotoxic.

FIG. 3A) is an exemplary illustration of an upconversion-mediated dynamic optogenetic response in 2 dimensions, which provides the model optogenetic system; HeLa cells express Cry2olig-mChy that oligomerizes and forms clusters in response to blue light.

FIG. 3B) is a set of exemplary microscopy images of cells expressing Cry2olig-mChy demonstrating oligomerization mediated by the TIPS-An:PdTPTBP upconversion emulsion, 10 min post stimulation with red light (1 s, 637 nm LED at 11 mW). Cells exposed to control emulsion (TIPS-An only) did not cluster in response to red light (637 am LED at 11-55 mW). The scale bars show 50 μm.

FIG. 3C) is an exemplary graph illustrating a correlation between the number of clusters observed and the concentration of the upconversion emulsion in the cell media (e.g., mean±s.d., n=12 sites per concentration). Quantification of the cluster ratio in cells, calculated by dividing the total cluster intensity by the total fluorescence of the whole cell.

FIG. 3D) is an exemplary plot of time-lapse results showing changes in cluster ratio (mean, n=12 sites per condition) and representative images (scale bar=25 μm), demonstrating reversibility of the optogenetic process.

FIG. 4A) is an exemplary confocal image providing an exemplary characterization of cell-laden TTA-UC hydrogels, e.g., of fibrin hydrogels with TTA-UC droplets, imaged by exciting with a red laser (637 nm) and detecting the upconverted emission of blue light (450 nm). Scale bar, 25 μm.

FIG. 4B) is an exemplary TTA-UC emission spectrum of the droplets imaged in A.

FIG. 4C) is an exemplary plot of a viability of cells encapsulated in the TTA-UC hydrogels including TIPS-An:PtTPTNP or TIPS-An:PdTPTBP emulsions, at various time points post encapsulation, normalized to hydrogels without emulsions (mean±SEM, n=9).

FIG. 4D) is an exemplary plot of a viability of cells encapsulated in the TTA-UC hydrogels at various time points post encapsulation. The emulsions contain TIPS-An:PtTPTNP, TIPS-An:PdTPTBP, TIPS-An (no sensitizer) or emulsions without chromophores (mean±SEM, n=9). The signal intensity increases over time due to proliferation of the cells within the hydrogels.

FIG. 4E) is a set of exemplary confocal images of cells encapsulated in the TTA-UC hydrogels, highlighting the distribution of the droplets and cells within the 3D matrix: HeLa cells (red, excitation 561 nm) and TTA-UC droplets (blue, excitation 405 nm). Top: cross-sectional image (x,y-plane, scale bar 25 μm.); Bottom: 3D rendering (x, y, z).

FIG. 5A) is an exemplary illustration of optogenetic responses from HeLa cells down to single-cell activation, with representative max intensity projections of confocal images of the HeLa cells encapsulated within the TTA-UC biomaterial two days post encapsulation: before irradiation and 10 min after irradiation with 637 nm laser excitation, showing Cry2olig-mChy photoreceptors oligomerize in response to the upconverted red-to-blue light from the TIPS-An:PdPTPBP system.

FIG. 5B) is an exemplary illustration of optogenetic responses from HeLa cells down to single-cell activation after irradiation with 710 nm scanning laser excitation, focused on a small cluster of cells (in red frame), showing response of cells to NIR-to-blue light from the TIPS-An:PtTPTNP system.

FIG. 5C) is a set of exemplary images providing an exemplary illustration of a representative experiment demonstrating single cell clustering in response to 710 nm excitation within TIPS-An:PtTPTNP TTA-UC hydrogel.

FIG. 6A) is an exemplary illustration of the process of TTA-UC, the process involves two chemical species, a sensitizer (Sen) and an annihilator (An). We use the notation ^{m}[Species]* to represent the multiplicity (m), which is either singlet (1) or triplet (3), and the excited state (*) or ground state (no asterisk) as an exemplary net process is

FIG. 6B) is an exemplary Jablonski diagram corresponding to the process of FIG. 6A), whereas the ground-state sensitizer ¹[Sen] is excited with low-energy light (hv); the singlet exciton ¹[Sen]* undergoes fast intersystem crossing (ISC) to its triplet state ³[Sen]*, which then undergoes efficient triplet energy transfer (TET) to the annihilator ³[An]*; and finally, two annihilator triplet excitons (³[An]*) can undergo triplet-triplet annihilation to yield one annihilator in the ground state ¹[An] and other in the singlet state ¹[An]* which then decays radiatively via fluorescence to emit a photon of high-energy light (hv).

FIG. 7A) is a set of exemplary diagrams of TTA-UC blue light annihilators illustrating absorption (black) and emission (blue/gray) spectra of TIPS-An and TTBP taken in dilute toluene.

FIG. 7B) is an exemplary illustration of chemical structures of TIPS-An and TTBP.

FIG. 7C) is an exemplary graph of a relative upconversion photoluminescence of TIPS-An and TTBP demonstrating that TIPS-An shows more intense emission in the 450-500 nm range, compared to TTBP.

FIG. 8A) is an exemplary set of diagrams of TTA-UC sensitizers providing the exemplary chemical structures of the two sensitizers used in this study: PdPTPBP and PtTPTNP.

FIG. 8B) is an exemplary normalized steady state absorption spectrum measured in dilute toluene, showing the intense Q-bands of the porphyrins: approximately 640 nm for PdTPTBP and 700 nm for PtTPTNP.

FIGS. 9A) to 9C) exemplary diagrams of a cell viability of emulsions with different reductant oils. Following 1 hr incubation with upconversion emulsions in different concentrations, CellTiter-Glo® Luminescent Cell Viability Assay was used to determine the number of viable cells in culture. FIG. 9A) showing exemplary graphs of the viability as a function of organic solvent contained within the droplets. FIG. 9B) showing exemplary graphs of the viability as a function of emulsion surfactants. FIG. 9A) showing exemplary graphs of the droplets containing soybean oil with and without the photon upconversion chromophores.

FIG. 10A) is an exemplary set of illustrations of Synthesis and Characterization of upconversion emulsion droplets, whereas an oil-in-water emulsion is generated by adding a mixture of an oil containing the annihilator and sensitizer chromophores to a buffer solution containing Pluronic F127 (9 wt %). FIG. 10B) Left side is an illustration of an exemplary maximum intensity projection of the emulsion droplets as imaged by confocal microscopy. The scale bar shows 25 μm. FIG. 10B) Right side is an exemplary graph of the exemplary size distribution of the droplets was measured by imaging processing, using particle analysis. Diameter was determined as the maximum Feret diameter of each droplet.

FIG. 11A) is an exemplary illustration of the exemplary solution UV-vis absorption spectra of the supernatant buffer collected from the hydrogels 24 h post formation (droplet mean diameter 250 n). The range of absorption wavelength 400-450 nm is characteristic of the annihilator TIPS-An, and the peak at 627 nm is characteristic of the sensitizer PdTPTBP. The result suggests that the droplets that are released to the buffer contain both the annihilator and the sensitizer.

FIG. 11B) is a set of exemplary diagrams of controlling release of the droplets from the hydrogel. Investigation of the effects of varying the percent of oil phase in the emulsions. As the amount of oil phase is increased, the resulting droplets are larger in size which enables to prevent release from the hydrogels. Exemplary size distribution in droplets as controlled by varying the percent of oil phase in the water phase of the emulsions. Dynamic light scattering was used for size measurements.

FIGS. 11C) and 11D) are exemplary illustration of how the supernatant buffer was further characterized by using dynamic light scattering (DLS), to confirm the presence of droplets in the buffer at 24 hours (see FIG. 11C)) and 72 hours (see FIG. 11D)) post hydrogel formation. The data shows the size of the droplets that are present in the supernatant, all the droplets are below the size of one micron.

FIGS. 12A) to 12C) are exemplary diagrams of an upconversion-mediated optogenetic response in 2D. FIG. 12A) is an exemplary set of illustrations when HeLa cells were directly irradiated using a pulse of blue light (λₑₓ=488 nm, 1 sec pulse), demonstrating that Cry2olig-mChy undergoes clustering in response to the high-energy stimulation. FIG. 12B) is an exemplary set of illustrations when the cell media containing TTA-UC emulsion (TIPS-An:PtTPTNP) was irradiated using a pulse of low-energy light (λₑₓ=710 nm, 1 sec pulse), demonstrating cell clustering in response to upconversion of the NIR light. FIG. 12C) is an exemplary set of illustrations when the cell media containing control emulsion (TIPS-An only) was irradiated using a pulse of low-energy light (λₑₓ=710 nm, 1 sec pulse), demonstrating lack of cell clustering in response to NIR light without upconversion. All images were obtained using the mCherry channel (λₑₓ=561 nm, and detection 570-640 nm) in a confocal microscope, clustering images were captured 10 min post irradiation. The scale bar shows 25 μm.

FIG. 13A) is an exemplary diagram of a design of upconversion biomaterial and cell encapsulation, which shows an exemplary synthetic method to produce the hydrogel biomaterial doped with upconversion droplets.

FIG. 13B) is an exemplary photograph of red light (λₑₓ=637 nm) penetration through the free-standing gel.

FIG. 13C) is an exemplary graph of an exemplary upconversion luminescence emission within the biomaterials upon excitation with red laser (λₑₓ=637 nm) detected using the spectral detector (400-580 nm) of a confocal microscope shows the blue emission within the upconversion biomaterial. Additionally, hydrogels can be imaged with the DAPI channel (λₑₓ=405 nm and detection 425-475 nm). The scale bars show 25 μm.

FIG. 14 is an exemplary diagram of a stability of the upconversion signal over time. Upconversion emulsions soybean oil and Pluronic F127 surfactant, and the TIPS-An/PdTPTBP sensitizer/annihilator pair, embedded into hydrogels, was continuously irradiated with red laser diode (640 nm) (1 mW) for 15 minutes without loose of signal.

FIG. 15A) is an exemplary photograph of to demonstrate the ability of red light (640 nm) to penetrate the hydrogels. TTA-UC hydrogel with dimensions of (z=2.5, x=1, y=1 cm) was made in a cuvette and photographed using a digital camera, with hydrogel under ambient light demonstrating a depth of 2.5 cm in the z axis.

FIG. 15B) is an exemplary photograph when Red LED (64 nm) was placed one cm away from one side of the hydrogel, to irradiate the hydrogel through the z axis under ambient conditions. Upconverted blue light was visible, and photographs show scattered blue light emanating throughout the hydrogel.

FIG. 15C is an exemplary photograph when the brightness of image B was reduced to show the penetrating gradient in the gel as a function of distance from the light source. FIG. 15D) Side view of the same hydrogel demonstrating the penetration of the light and the brightness of the hydrogel.

FIG. 15E) is an exemplary photograph of a free standing hydrogel upconversion upon red light excitation, demonstrated by taking the hydrogel out of the cuvette and placing it on a glass slide. Photographs were taken by using a cutoff filter in front of the lens of the digital camera.

FIG. 16 is an exemplary diagram of imaging cells within the upconversion biomaterials 4 days post encapsulation. Representative confocal microscopy image of a cluster of live cells, 96 hr post encapsulation in the upconversion biomaterials using TIPS-An:PtTPTNP emulsion droplets. The scale bar shows 25 μm.

FIGS. 17A) and 17B) are exemplary diagrams of control groups for triggering cells in 3D with upconversion biomaterial. Representative max intensity projection of confocal image of the cells encapsulated with the control hydrogel, TIPS-An only with not sensitizer, two days post encapsulation before irradiation and 10 minutes after irradiation with: FIG. 17A) providing 637 nm laser excitation, showing no clustering of Cry2oligo-mCherry photoreceptors, and FIG. 17B) showing 710 nm scanning laser excitation, focused on a small cluster of cells (in red frame), showing no response of cells.

FIGS. 18A) to 18C) are exemplary diagrams of an exemplary stimulation of cells 1-day post encapsulation, with FIG. 18A) showing microscopy imaging of cells (red) and microdroplets (blue), with FIG. 18B) providing an illustration before 710 nm laser stimulation, and FIG. 18C) providing the exemplary illustration after 710 nm laser stimulation demonstrating Cry2oligo-mCherry clustering. Projection in the z-axis. Scale bar 25 μm.

FIGS. 19A) to 19C) are exemplary diagrams of a stimulation 3 days post cell encapsulation, whereas FIG. 19A) is an illustration of an exemplary microscopy imaging of cells (red) and microdroplets (blue), and FIG. 19B) is an exemplary illustration of mCherry signal before and after 710 laser stimulation, demonstrating Cry2olig-mCherry clustering. Projection in the z-axis. Scale bar 25 μm.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The ability to probe cells with high spatiotemporal resolution is critical to improving fundamental understanding of cellular function and developing new disease therapies and diagnostics. However, current techniques involve proteins/chromophores triggered by visible light (e.g. blue light), which does not penetrate well into tissue. In optogenetics, cells are genetically modified to contain light-responsive proteins. Most optogenetic proteins are triggered by high-energy blue light, but blue light does not penetrate deep tissue. Lower-energy light (e.g. red, infrared, near-infrared) is needed to probe deep tissue and capture 3D structural complexity.

The present disclosure utilizes upconversion biomaterials to stimulate optogenetic proteins in deep 3D scaffolds. Chromophores capable of converting low-energy red light to high-energy blue light may be embedded in biocompatible hydrogels. This enables utilization of red light to stimulate deep tissue using non-cytotoxic materials. This invention enables more selective stimulation of optogenetic proteins in deep tissue and has the potential to provide critical insights into cell function and disease.

Optogenetics is a technique that utilizes light to manipulate cellular activity with high spatiotemporal control [S-1]. Optogenetic cells are genetically engineered to produce light-sensitive proteins that may be triggered by different wavelengths of light [S-1]. High-energy blue light is absorbed or scattered at the surface [S-2]. Lower-energy red light penetrates further to reach deep tissue, but often lacks the energy required to trigger optogenetic proteins [S-2]. There is a need for developing biocompatible waveguides to selectively deliver light to deep tissue and illuminate 3D structural complexity [S-3]. Hydrogels are biomaterials capable of selectively delivering light to optogenetic proteins [S-3]. Upconversion is a process by which two low-energy photons are converted to one high-energy photon [S-4]. Upconversion biomaterials are emerging therapeutic agents and imaging probes [S-4].

The exemplary embodiments of the present invention provide a new class of 3D biomaterials for optogenetics. According to an exemplary aspect of the present disclosure, chromophores capable of upconversion may be embedded into a hydrogel to convert low-energy light, such as red light and near-infrared light, to higher-energy light, such as blue light. The exemplary embodiments of the present disclosure can facilitate a stimulation of optogenetic proteins in deep tissue. The exemplary embodiments of the present invention may be used with various kinds of cells, such as multipotent and pluripotent stem cells (SC). The materials and systems according to the exemplary embodiments of the present invention may use biocompatible and non-cytotoxic materials. The exemplary materials, systems and methods according to the exemplary embodiments of the present disclosure may improve spatiotempoml resolution of stimulation, may provide insight into fundamentals of cell function, may provide means to 3D printing, may provide the means to trigger photochemical processes within the biomaterials, and may elucidate disease mechanisms and enable development of related therapeutics.

In native tissues, cells are subjected to precisely orchestrated signals from the environment, which have profound outcomes on tissue morphogenesis, homeostasis, and disease.[1] The interactions between cells and their environment revolves around cellular signaling processes at steady-state in space and time to dictate their fate: proliferation, migration, and differentiation. To sense these cues, cells use various signaling networks, and behavioral changes follow after specific interactions occur.

Mimicking signaling events in 3D is a critical challenge. Recapitulating the dynamic behavior of signaling networks with fine spatiotemporal control is particularly challenging. According to exemplary embodiments of the present disclosure, it has been recognized that the use of light to stimulate cell-signaling pathways could lead to unprecedented precision in the control of cellular behaviors.[2] The key to developing this methodology may involve fusing light-sensitive proteins to various extracellular and/or intracellular proteins that can be activated or deactivated by light. Today, most of the optogenetic proteins are activated by blue light which cannot penetrate deep into 3D tissues, thus limiting the success of this technology/approach. The exemplary embodiments of the present disclosure relate to providing a novel biomaterial that can control cell behavior in 3D using low-energy light with unprecedented specificity and dynamic perturbation. Harvesting light-responsive human cells in a biomaterial providing conversion of infrared light into blue light may allow precise spatiotemporal control of cell function for tissue repair and disease control.

The exemplary embodiments of the present disclosure can relate to an unconventional, yet modular and user-friendly technology to manipulate cell signaling with exquisite spatiotemporal control in 3D, using optically active biomaterials that can benefit regenerative medicine and tissue repair. Specifically, provided is a process that converts low-energy light into high-energy visible light triplet-triplet annihilation upconversion (TTA-UC)—in order to controllably trigger light-responsive proteins. There may be inherent limitations found in current photon upconversion mechanisms at the conditions necessary for cell survival. Thus, successful demonstration of TTA-UC in 3D biomaterials is especially significant because the methodology introduces a new way to activate optogenetic cells using light to control their behavior deep within the biomaterials, with precise control in time and space.

In its most fundamental dimension, an important question can be: how can low energy near infrared (NIR) light be interfaced with biomaterials to trigger optogenetic proteins within cells in 3D? A major challenge may be that most optogenetic proteins are activated by blue light (˜490 nm), and such photons cannot deeply penetrate living tissues and activate the cells in 3D environments due to light scattering, background absorption, competing absorption by endogenous chromophores, etc. Other optical techniques require highly specialized equipment and only lead to localized excitations.[3]

To overcome this challenge, according to exemplary embodiments of the present disclosure, new classes of modular chromophores can be provided that can convert low energy infrared near-infrared (NIR) light to visible light. This is a key feature for optogenetic applications since the NIR optical window (e.g., 650-1350 nm) has its maximum tissue penetration depth with minimal damage to the cells. Starting from this major advancement, this disclosure provides engineer photon upconverting 3D biomaterials that accommodate a cell expressing a light activated protein, cryptochrome 2.

As used herein, “near-infrared light” or “NIR light” includes light having wavelength in a range of from about 700 nm to about 1400 nm, for example, 700 m to about 750 nm, from about 750 nm to about 800 nm, from about 800 nm to about 850 nm, from about 850 nm to about 1000 nm, or from about 1000 nm to about 1400 nm.

As used herein, “red light” includes light having wavelength in a range of from about 600 nm to about 750 nm, for example, from about 600 nm to about 650 nm, from about 650 nm to about 700 nm, or from about 700 nm to about 750 nm.

As used herein, “blue light” includes light having wavelength in of range of from about 400 nm to about 500 nm, for example, from about 400 nm to about 450 nm, or from about 450 nm to about 500 nm.

The exemplary embodiments of the present disclosure can provide a new class of biomaterials capable of converting red light into blue light and to enable stimulation of optogenetic cells in 3D settings. Low-energy NIR light may allow on-demand external activation of signaling pathways with spatiotemporal precision, because tissue is relatively transparent to NIR light.[4,5] Moreover, implementing upconversion chromophores to function under ambient conditions has been an obstacle [6] for interfacing these systems with living organisms.

According to the exemplary embodiments of the present disclosure, it is possible to obtain blue light from NIR in hydrogels, in the presence of cell culture media, at ambient conditions. In some aspects of the present disclosure, select upconversion materials is combined with encapsulating cells in a 3D hydrogel matrix, in order to produce a system in which upconverted light can be used to activate optogenetic cells.

The widely accepted tenets to control cell behavior for regenerative therapeutics are two pronged—soluble signals, such as growth factors and small molecules, and extracellular matrix (ECM) mimics [1,7]. While it may be vital to understand the intricacies of their roles in order to control signal transduction, neither can be manipulated externally, site-specifically, and on-demand. The present disclosure may provide the ability to employ unconventional photophysical light-energy harvesting processes to stimulate cell behavior with external control. The exemplary embodiments of the present disclosure implements two fundamentally unique and innovative concepts. (i) adapting the concept of triplet fusion upconversion (or TTA-UC) [8-10] from organic materials to facilitate deep penetration and provide high-energy visible light photons or NIR light photons from within the biomaterials (for example, NIR photon upconversion to visible [blue] light); and (ii) controlling the activation and homogeneous control of optogenetic cells using light, within 3D biomaterials.

The 3D biomaterials of the present disclosure that are activated with NIR light may open new areas of exploration in the development of medical devices, and drug delivery, among other applications. To date, key challenges in the field of TTA-UC optogenetics in bioengineering remain in the development of cell-compatible optical systems.[11,12]. Specifically, triplet fusion upconversion, the photophysical process responsible for TTA-UC, requires two chromophores, one for light harvesting and the other for exciton upconversion [13]. These exemplary materials are commonly explored for solar energy conversion in photovoltaic devices, and are likely to be cytotoxic. Moreover, the process were mainly demonstrated in organic solvents and in the absence of oxygen (oxygen quenches the triplet-state excitons formed upon light absorption) [6]. Finally, the ability to spatiotemporally control cell behavior in 3D remains a major challenge. Strategies to finely tune key signaling pathways in cells are limited and based on chemically modifying the entire (synthetic) matrix [14-16], or by adding chemical signals to the medium [1]. The TTA-UC hydrogels may address longstanding questions about signaling in developmental biology research in 3D.

### Exemplary Approach

Some modes for carrying out the exemplary embodiments of the present disclosure are presented in terms of its aspects, herein discussed below. However, the present invention is not limited to the described exemplary embodiment and a person skilled in the art will appreciate that many other embodiments of the present invention are possible without deviating from the basic concept of the present invention, and that any such work around will also fall under scope of this application. It is envisioned that other exemplary styles and configurations of the present disclosure can be easily incorporated into the teachings of the present invention, and only one particular exemplary configuration shall be shown and described for purposes of clarity and disclosure and not by way of limitation of scope.

Headings used herein are for organizational purposes only and are not meant to be used to limit the scope of the description or the enclosed claims. As used throughout this application, the word “may” is used in a permissive sense (i.e., meaning having the potential to), rather than the mandatory sense (i.e., meaning must). The terms “a” and “an” herein do not denote a limitation of quantity, but rather denote the presence of at least one of the referenced items.

### Exemplary Strategy

First, described below is an example of a method of designing and synthesizing 3D biomaterials [26] containing encapsulated chromophores that can undergo photon upconversion, converting infrared light into blue light in order to provide the appropriate energy to the stem cells for differentiation. Second, described below is the introduction of optogenetic cells to the infrared upconverting 3D scaffolds to test the ability to activate them using NIR light [27]. In an exemplary aspect of the present disclosure, the TTA-UC hydrogels are non-cytotoxic and capable of converting low-energy NIR photons into visible-light, high-energy photons ranging from yellow to blue light. In an exemplary aspect of the present disclosure, the TTA-UC 3D scaffolds is interfaced with optogenetic cells and maintain cell viability over time. In an exemplary aspect of the present disclosure, the photoactivation of signal transduction is characterized and monitored.

### Photon Upconversion 3D Biomaterials

The present disclosure provides hydrogels that are doped with micelles [28] of triplet fusion upconversion chromophores, which absorb NIR light and emit visible light to deliver the high-energy photons to the photo-responsive cells incorporated within the biomaterial. Varieties of molecules that undergo triplet fusion upconversion exhibiting remarkably large anti-Stokes shift and tunable color emission are disclosed in References (8) to (10), the disclosures of which are hereby incorporated by reference in their entireties.

The exemplary embodiments of the present disclosure can provide the fabrication and characterization of modular non-cytotoxic 3D biomaterials containing triplet fusion upconversion chromophores that emit blue (or visible) light upon excitation with NIR light. For example, the organic triplet fusion upconversion chromophores may be integrated in a variety of micelles/emulsion droplets/particles that are loaded within, e.g., fibrinogen hydrogels, biopolymer-derived hydrogels, synthetic hydrogels, polymeric ionic liquid hydrogels, or combinations thereof. The composition, microstructure, and mechanics of the biomaterials may be characterized. Preferably, the triplet fusion upconversion chromophores do not degrade in the presence of oxygen and leaching of the active chromophores from the micelles to the environment is prevented.

Photon Upconversion Mediated by Triplet Fusion within Micelles

The chromophores used in the exemplary embodiments of the present disclosure may be tuned such that the sensitizer-annihilator pair may obtain the desired output of light with a wavelength of about 488 nm upon excitation with NIR light. In certain exemplary embodiment of the present disclosure, the output of light may have a wavelength in a range of about 390 nm to about 550 nm, or about 390 nm to about 450 nm, or about 400 nm to about 500 nm, or about 450 nm to about 550 nm.

The upconversion components may be tuned to adjust the output wavelength, accessing both orange-to-blue light from low-energy NIR light.[8-10]. The process may involve two chromophores, the sensitizer (low-energy light absorber) and the annihilator (low-energy light emitter). Traditionally, the upconversion molecules are dissolved in organic solvents because the hydrophobic sensitizer and emitter dyes have limited solubility in water. However, for biomedical applications, only aqueous systems can be used. To achieve this goal, the upconversion chromophores may be encapsulated within micelles [28]. For example, the chromophores may be encapsulated within micelles. In order to finely adjust the output light emission, other types of sensitizers and annihilator chromophores may be used, such as diketopyrrolopyrrole⁸, anthracene²⁹ and tetracene⁹ derivatives, and other chromophores that undergo TTA-UC. Examples of the chromophores that may be used in the present invention are disclosed in References (8), (29) and (9), the disclosures of which are hereby incorporated by reference in their entireties. The micelles may be made using at least one of the following surfactants: Pluronic F127, Tween 80 and Triton X-100. The resulting upconverting micelles are stable, bright, and can be dispersed in water, buffer, or cell culture media. Such modularity may allow running various controls in order to understand the important mechanisms to activate optogenetic cells.

The sensitivity of upconversion micelles to the presence of dioxygen in living cells and biological tissues represents a challenge. To overcome this challenge, a natural additive, such as soybean oil [30], may be added into these micelles. This natural oil has been recently shown to locally deplete the concentration of oxygen in the micelles and enable stable upconversion in aerated environments. The upconversion efficiency at ambient conditions may be quantified in order to optimize the process and ensure that prolonged irradiation does not quench upconversion.

### Triplet-Triplet Annihilation Upconversion (TTA-UC) Biomaterials

There are two exemplary aspects in the design of stable TTA-UC biomaterials: 1) stability of upconversion under prolonged irradiation; and 2) maintaining the integrity of the mechanical properties of the doped gels. In an embodiment of the present invention, the hydrogel may contain fibrin gels. The production of visible light following excitation with NIR light of fibrin gels containing TTA-UC micelles may be detected by confocal microscopy. In order to modulate the intensity of the upconverted light output, the type of surfactants used and the loading amounts may be varied, while monitoring changes to the mechanical properties of the gels by dynamic mechanical analysis (DMA).[31].

TTA-UC0 fibrin hydrogels may be made by mixing micelles with fibrinogen solution before adding the thrombin cross-linker. The micelles in the gel may be synthesized by a modified route adding the upconversion chromophores to soybean oil.

The micelles may leach the chromophores over time, which may also be toxic to cells. Therefore, the extent of leaching may be monitored, and if issues arise, the micelles may be further protected by encapsulation in a layer of silica [32] to provide additional stability. This strategy may also serve as another barrier to prevent oxygen quenching. The materials and/or systems may be characterized by optical microscopy and their stability may be monitored following prolonged irradiation at ambient conditions. The micelle size-dependence may also be evaluated by comparing the performance of sub-micrometer micelles with emulsions and macroparticles, using various surfactants and oil-in-water dispersions [33].

With the successful incorporation of various TTA-UC chromophores dispersed in micelles within the hydrogels, the incorporation of cells within these 3D biomaterials may be investigated. However, it may be possible that the upconverting micelles may experience burst release due to changes in concentration, which may impact the operating concentration above/below the critical micelle concentration (CMC). To prevent such problems, the types of surfactants may be varied. In certain embodiments, oil-in-water emulsions [34] may be used. The biomaterials may be thoroughly characterized by optical microscopy, DMA, and cryo-scanning electron microscopy to understand the microenvironment where the cells reside.

### Characterize and Validate the Ability of TTA-UC Biomaterials to Activate Optogenetic Cells.

Triggering cell signaling pathways on-demand within 3D biomaterials has the great potential to modulate cell behavior with unprecedented spatiotemporal control. In order to achieve such precision control, optogenetic cells may be incorporated with the TTA-UC biomaterials. Specifically, the biocompatibility of the biomaterials, with attention to the TTA-UC systems that are loaded within, may be characterized. For example, this new optogenetic technique combined with directed illumination of modified photoresponsive cells expressing a light-activated signaling pathway may be characterized. Various upconversion chromophores that emit specific wavelengths of light may be used and characterized to understand the energetic requirements to activate optogenetic proteins. Additionally, low-energy light-penetration experiments may be carried out, varying the depth of the cell-loaded biomaterials to investigate the depth of penetration of the NIR light capable of activating optogenetic cells. These studies may be compared to direct stimulation with visible light diodes, excluding the TTA-UC chromophores to quantify the deep light penetration and efficiency in activating, e.g., modified photoresponsive cells, deep within the biomaterials.

### Biocompatibility of TTA-UC Biomaterials

Given that the biomaterials comprise hydrogels that are doped with potential toxins, various types of cells may be encapsulated within the TTA-UC hydrogels (e.g., modified photoresponsive cells, fibroblasts, endothelial cells, or combinations thereof). A requirement for the TTA-UC hydrogels is biocompatibility, which may be tested by live/dead assays. In addition, actin staining may be performed followed by confocal imaging to evaluate the ability of the cells to spread and reorganize within the hydrogel. For example, a surfactant for constructing the micelles, F127, is biocompatible.

### Exemplary Characterization of Unconverted Light Intensity and Penetration Depth

Characterization of the upconverted light generated post cell encapsulation may enable to determine the efficiency of the TTA-UC biomaterial in terms of (i) intensity of the upconverted light transmitted to the encapsulated cells (ii) penetration depth and determination of size limitations of the biomaterial. For example, 1×10⁶ cells may be seeded within 100 μL of TTA-UC biomaterial in 3D. The biomaterials may be incubated for 3 days prior to light stimulation to allow the formation of cell-cell interactions. Confocal microscopy may be used to determine the relative intensity of the upconverted light generated from biomaterials at various depths. Image analysis tools may enable to compare the relative intensities at different penetration depth within. While a single particle or a cluster of particles can efficiently generate upconverted light, the overall intensity of the light within the biomaterial may be insufficient to activate optogenetic proteins post cell encapsulation due to (i) light scattering as it travels through the gel (ii) the low concentration of the upconversion solution in the matrix. In order to minimize light scattering, the size of the micelles may be optimized from micrometers to nanometer by using different surfactants with different concentration. The concentration of the micelles may be optimized to achieve a balance between biocompatibility and upconversion efficiency.

To test for penetration depth, TTA-UC hydrogels containing fibroblasts may be prepared with different thicknesses: for example, 100 μm, 250 μm, 500 μm, 1 mm and 2 mm. The gels may be placed on top of optogenetic endothelial cell monolayers and stimulated by LED (640 nm) positioned above the hydrogel. Ten minutes post stimulation, activation of the optogenetic proteins may be determined by clustering of the construct on the cell membrane. This experiment may enable to determine the threshold for penetration depth of the NIR capable of generating enough energy to activate the cells.

As a first step toward TTA-UC optogenetics in 3D, according to the exemplary embodiments of the present disclosure, it is possible to stimulate Cry2olig-mCherry cells using a 640 nm laser in 2D, using upconversion chromophores dispersed in soybean oil. Cells were cultured as monolayers and the upconversion oil was admitted on top of the cells prior to stimulation with red light. Next, the cells were stimulated with a 640 nm laser for 10 minutes.

The biocompatibility of the TTA-UC hydrogels may depend on micelle concentration. While low concentration of micelles has demonstrated cell survival over time, higher concentrations may hamper cell survival. To tackle this challenge, the type of TTA-UC encapsulation system may be changed, such as using silica-coated nanoparticles and soybean oil-in-water emulsions. These alterations may decrease leaching of the upconversion chromophores, which may be the source of toxicity.

Additionally, the potential challenges of working in the 3D hydrogel are (i) light scattering as it travels through the gel and (ii) the concentration of the TTA-UC chromophores in the matrix may lead to low light intensity, incapable to activate the optogenetic protein. Light scattering is generally an issue with high energy photons. The size of the 3D biomaterials loaded with cells may be varied. Using confocal microscopy, optimal depths that achieve light penetration may be characterized by looking into the homogeneity of the cells that contain clustering. The type of TTA-UC chromophores may be varied to vary the light-energy output. Varying the wavelength of emission may reduce background absorption and scattering. Similarly, the concentration of the micelles may be optimized by changing the oil-to-water ratio, to achieve a balance between maximum biocompatibility with maximum upconverted photon intensity. It was determined that light intensity of upconverted photons is not an issue in chemical transformations that generally require high photon flux of visible light [10].

### Exemplary Synopsis and Outlook

The ability to control cell signaling in 3D with light represents a powerful tool to study cell behavior in 3D. This disclosure provides biomaterials that combine TTA-UC chromophores within hydrogels containing optically responsive cells. The 3D biomaterials may efficiently upconvert NIR light to tunable visible light, such as blue light. The upconverted light may be used to trigger signaling pathways in cells engineered with light-responsive proteins.

Applications of the materials, systems and methods of the present invention may include the following: research tools for studying cell regulation and misregulation; research tools for studying neurodegenerative disease; potential diagnostic and/or therapeutic tools for neurodegenerative diseases; potential biomaterials for targeted drug delivery; integration of chromophores and hydrogels into biodegradable electronics: and biosensors.

Optogenetics has emerged as a powerful technique to manipulate fundamental cell signaling processes with spatiotemporal control using light as an external trigger. However, the ability to manipulate optically-induced biological responses in 3D has been dwarfed by the physical limitations of visible light penetration to trigger photochemical processes. Given that many biological systems are relatively transparent to low-energy light, according to exemplary embodiments of the present disclosure, hydrogel biomaterials can be provided that are capable of converting near-infrared (NIR) light into blue light within the cell-laden 3D scaffolds. The resulting upconverted light can then be harnessed by optically active proteins in cells to trigger a photochemical response. The modus operandi of the biomaterials is based on triplet-triplet annihilation upconversion (TTA-UC). We observe that the biomaterials are capable of triggering an optogenetic response by long-wavelength irradiation (NIR-to-blue TTA-UC) of HeLa cells that have been engineered to the express the blue-light sensitive protein Cry2olig. While it is remarkable to photoinduce the clustering of Cry2olig with blanket NIR irradiation in 3D, it was also demonstrated how a single cell can be activated with great specificity and spatiotemporal control. These biomaterials enable the ability to photochemically control cell signaling processes and cell fate within 3D scaffolds, which can lead to a wealth of fundamental studies.

According to exemplary embodiments of the present disclosure, photo-active proteins in a single cell can be activated with great specificity by focusing a beam of irradiation of the TTA-UC droplets on the vicinity of the single cell. According to exemplary embodiments of the present disclosure, a user-selected pattern of spatiotemporal control of activation of a photo-active protein in a cell can be achieved. According to exemplary embodiments of the present disclosure, instead of a pre-determined pattern of control, the pattern of control may be adjusted during or after starting irradiation of light.

The ability to modulate cell function through on-demand external triggers within three-dimensional (3D) biomaterials can significantly transform biological studies on chemically induced signaling pathways, impacting areas of disease control/prevention, and tissue engineering^{1,52,53}. Specifically, light-responsive biomaterials provide newfound opportunities for fundamental studies in photochemical processes within synthetic matrices that mimic the native microenvironment of cells^{2,3}. The use of light to induce photochemical processes offers numerous advantages since it can be readily modulated in space and time. To date, light-activated reactions that require short-wavelength irradiation in the visible region (λ<500 nm) intracellularly, or in the cell microenvironment, have been mainly carried out in 2D cultures, such as polymerizations from cell surfaces⁴ and optogenetic processes⁵⁻⁷. In 3D, however, there are prohibitive limitations when inducing photochemical transformations within 3D matrices. A major obstacle lies in the need to use blue-light to induce reactions, since high-energy photons have short penetration depth through most solid-state media (e.g., cell-loaded 3D biomaterials, tissue etc.) due to light scattering and background absorption⁸. To overcome such challenges, processes that involve long-wavelength two-photon excitation have been applied to various systems, albeit with costly limitations in the use of high fluence equipment and small surface area of irradiation^{9,10}. Alternatively, recent breakthroughs empoly unconventional photophysical processes where materials that absorb low-energy photons in the infrared region and emit high-energy photons (photo upconversion) that can be exploited for light stimulation of neurons¹¹⁻¹³.

Considering the potential impact of upconversion systems, the non-linear photophysical process of photon upconversion with inorganic nanoparticles (UCNPs)^{11,12,14} or by the multiexciton process of triplet-triplet annihilation upconversion (TTA-UC) from organic chromophores have the potential to be exploited in synthetic biomaterials due to the low power excitation required and tunable excitation/emission wavelengths¹⁵⁻¹⁷. By implementing photon upconversion processes that operate by blanket and/or focused irradiation using inexpensive commercial low-energy light sources, the realization of photochemically-induced processes in 3D can enable a wide variety of studies of biological systems^{18,19}. For example, a key strategy for manipulating cells with light involves fusing optogenetic proteins to various cellular proteins that can be either activated or deactivated by blue light^{6,20}. Thus, the ability to integrate upconversion systems within biomaterials provides a route to exquisitely control photochemically triggered processes in 3D cultures and through tissue penetration (drug delivery, degradation, cell signaling, etc.). In this vein, according to exemplary embodiments of the present disclosure, it is possible to focus on the development of biocompatible TTA-UC biomaterials, e.g., for 3D cell cultures.

While photon upconversion in biology has been mainly focused on using lanthanide-based inorganic nanoparticles^{11,12}, TTA-UC has received less attention in optogenetics^{13,21} mainly due to challenges associated with biocompatibility, sensitivity to oxygen, and insolubility of the organic chromophores in aqueous media, among other issues^{22,23}. In most cases, nanoparticles containing TTA-UC chromophores have been used for drug delivery and imaging²⁴.

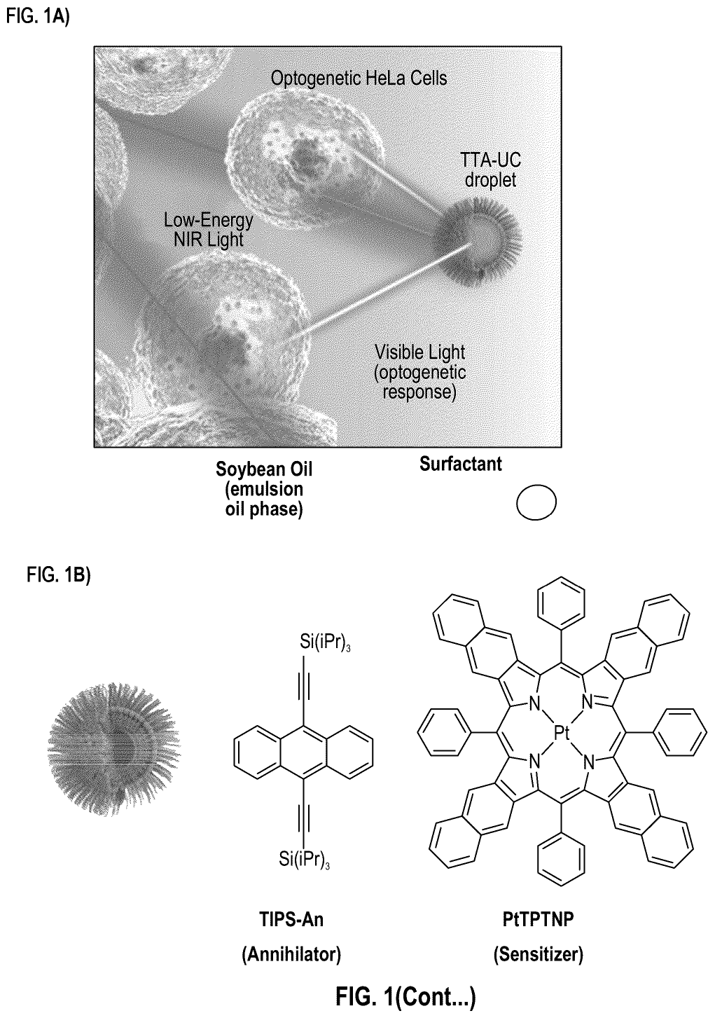

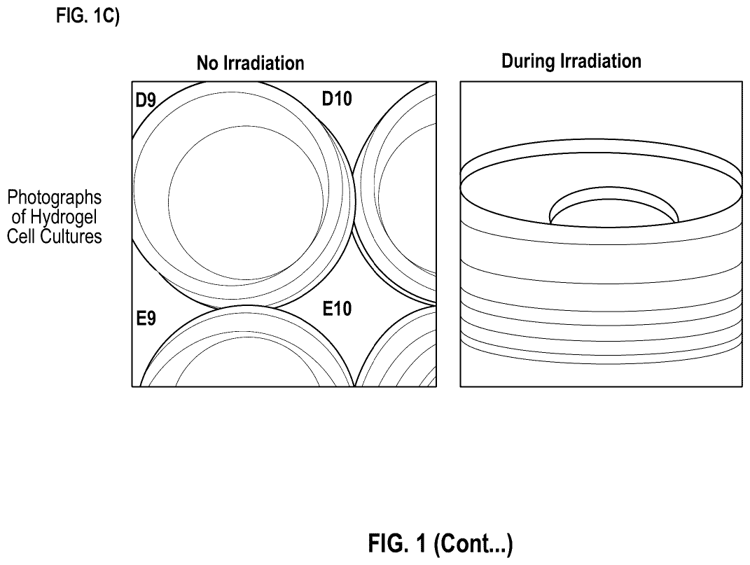

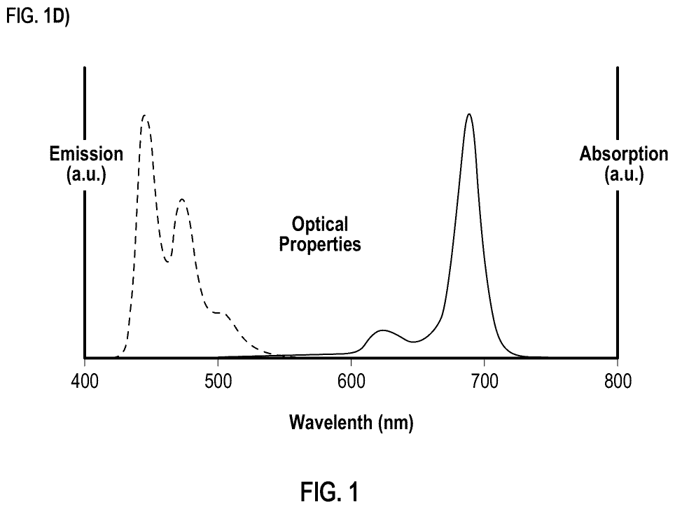

Considering the challenges and vast potential applications of TTA-UC 3D biomaterials, here, a materials platform of biocompatible hydrogels that are capable of absorbing near-infrared (NIR) light (λ˜700 nm) and emitting blue light (λ<500 nm) within the 3D scaffold can be provided. As proof-of-principle, the ability to photochemically trigger a primary optogenetic response can be facilitated using HeLa cells expressing a variant of the optogenetic Arabidopsis photoreceptor Cryptochrome 2 (Cry2olig). The general concept is shown in FIGS. 1A and 1B, where the oil-in-water TTA-UC emulsion droplets are formed with a surfactant that encapsulates the sensitizer chromophore (NIR light absorber) and the annihilator (visible-light emitter) in soybean oil. The absorption and emission spectra of the two systems are also shown in FIG. 1D to highlight the apparent anti-Stokes shift. Hydrogels are then formed, containing the HeLa cells expressing Cry2olig and the TTA-UC emulsion droplets under aerobic conditions in buffer. The resulting biomaterials are visibly opaque due to light scattering, but under blanket irradiation with NIR light, the hydrogels internally emit blue light (FIG. 1C). The system is modular, as different combinations of TTA-UC chromophores and the hydrogel support network (e.g., synthetic or biopolymer derived hydrogels) can be readily modified.

### EXAMPLES

### Exemplary Methods

### Exemplary Materials

Unless otherwise stated, all starting materials were obtained from commercial sources and used without further purification. PdTPTBP was purchased from Santa Cruz Biotechnology and was used without further purification. PtTPTNP² and TTBP³ were synthesized according to reported procedures. TIPS-An was synthesized as described below.

### Exemplary Synthesis of TIPS-Anthracene

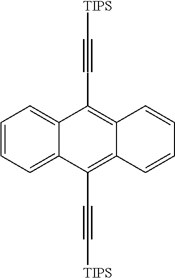

To a solution of TIPS-acetylene (2.7 g, 15 mmol) in anhydrous THF (15 mL) under argon at 0° C. was added n-butyl lithium (6 mL, 2.5M in hexane) over 5 min. The reaction was allowed to come to RT. After 1 h, anthraquinone (1 g, 5 mmol) was added in one portion and the reaction stirred overnight. The reaction was quenched and diluted with wet THF and poured into a rapidly stirred solution of stannous chloride dihydrate (33 g, 0.15 mol) in 10% hydrochloric acid (75 mL). After 1 h, the reaction was extracted with DCM (×3), the combined extracts washed with water (×3), brine and dried over MgSO₄. The solution was finally filtered through an inch of celite to remove residual stannous impurities. The residue obtained after concentration in vacuo was purified by column chromatography on silica gel eluting with hexane to give the title compound at a bright yellow solid (2.3 g, 84%).

¹H NMR (500 MHz, CDCl₃) δ 8.67 (dd, J=6.7, 3.2, 4H), 7.63 (dd, J=6.7, 3.2, 4H), 1.34-1.25 (m, 42H)

### Exemplary Synthesis of UC Emulsions

To a glass tube were added 100 μl of oil (soybean oil/D-limonene/TMB) and 900 ul of HEPES buffer containing Pluronic F127 (10% v/v). The oil/aqueous buffer mixture was first gently agitated and then homogenized using a rotor-stator or a bead miller homogenizer. If the emulsion was not to be cultured with cells, then the resulting oil-in-water emulsion was used directly without further processing. If the emulsion was to be cultured with cells then the resulting emulsion what filtered through a 0.22 um syringe filter in sterile conditions to produce a sterile emulsion. For TTA-UC emulsions, the sensitizer/annihilator were first dissolved in chloroform and then mixed to the oil (10% v:v). The chloroform was evaporated by stirring the emulsion with an open cap for several hours.

### Exemplary Synthesis of UC Hydrogels

Fibrin scaffolds were made from bovine or human fibrinogen and thrombin. Fibrinogen (20 mg/mL, 0.5 mL) (Sigma) was dissolved in HEPES buffer and filtered through a 0.22-lm low-protein binding filter. The fibrinogen solution or the cell line mixed in was poured into wells of a 96-well culture plate. TTA-UC or control emulsion was added to the well at different concentrations. Then, the thrombin solution (5 U/mL, 0.5 mL)(Sigma) was poured into each well to form the fibrin scaffold.

### Exemplary Synthesis of Cell-Laden Upconversion Hydrogels

Hydrogels were formed in 96-well plates, for each well: CRY2olig-mChy stable HeLa cells were counted and 50,000 cells were resuspended in 35 μl Fibrinogen from human plasma (at 20 mg/ml; Sigma F3879), supplemented with 2.5 μl CaCl₂ at 50 mg/ml (2.5 mg/ml final concentration) and the indicated volume of emulsion. If needed, volume was adjusted to 40 μl with HEPES buffer (Gibco). The 40 μl, containing cells, fibrinogen and emulsion droplets was then added to a 96 well, containing 10 μl of Thrombin from human plasma (equals 0.25 units; Sigma T6884) and gently mixed with a pipet three times to form the hydrogel. Hydrogels were incubated for 30 min in 37° C. before adding 100 μl growth media supplemented with protease inhibitor cocktail (1:200; Sigma P1860) to avoid digestion of the hydrogel by the cells' proteases.

### Exemplary Characterization of Size of Oil Droplets

Particles were imaged on a Nikon A1RMP laser scanning system based on an Eclipse TiE stand equipped with a 40×/1.3 PlanFluor oil-immersion objective. Excitation was at 405 nm and emission was collected from 425-475 nm. Scan zoom was set to give a pixel size of 0.316 um. Z series were collected over a depth of 11 μm with a step size of 1 μm.

For analysis, maximum projections were generated and locally thresholded for particle analysis using Fiji. The Bernsen algorithm was used with a radius of 5 and a local contrast threshold of 30, and a binary watershed was performed to separate adjacent particles. Diameter was determined as the maximum Feret diameter of each particle.

### Exemplary Cell Culture

HeLa cells (obtained from American Type Culture Collection) were maintained in Dulbecco's Modified Eagle's Medium (Gibco) supplemented with 10% fetal bovine serum (Invitrogen) at 37° C. in a humidified 5% CO₂ atmosphere.

### Stable HeLa Cell Line

To generate the stable HeLa cells, constructs containing the CRY2oligo-mChy within the piggyBac transposon were introduced into cells by transfection and integrated using the piggyBac transposase.³⁷

### Exemplary Cell Viability Assay

For viability assays testing biocompatibility of the emulsions, cells were plated in a 96-well white opaque plate format at 40,000 cells per well, allowed to seed overnight, and then treated with emulsions at indicated concentrations for one hour. These viability assays utilized the Cell-Titer-Glo 2.0 reagent (Promega, G9243) according to the manufacturer's instructions. Briefly, 100 μL of Cell-Titer-Glo 2.0 reagent, diluted at 1:1 with culture media, was added to each well containing 100 μl emultion-particles-containing media. Plates were gently agitated for 2 minutes and incubated at room temperature for 10 minutes in the dark to promote adequate mixing. Luminescence was subsequently measured using a Victor 5 plate reader (Perkin Elmer). The intensity of luminescence was normalized to that of cells with no emulsion particles added.

Cell viability in 3D hydrogels was measured in the same manner.

### Exemplary Photo-Stimulations and Fluorescent Microscopy in 2D

Images for were taken on an Axio Observer 7 microscope (Zeiss) using 20× objective (0.8 N.A) with 2-by-2 pixel binning. For live-cell imaging, cells were for imaged a total of 70 minutes in one-minute intervals in a humidified and 37° C. chamber in 5% CO₂. Stimulation with red light was for a total of 1 second. Total light exposure time for mCherry images was kept at 200 ms for each time point. Groups are n=12 frames. Images were processed using a Matlab script. Briefly, a mask of puncta was generated by top hat-filtering raw images with a circular kernel of radius 4 μm and thresholding absolute intensity. The puncta parameter represents the number of puncta in the field of view.

### Exemplary Confocal Microscopy in 3D

Hydrogels were imaged and irradiated on a Nikon A1RMP laser scanning system based on an Eclipse TiE stand equipped with a 40×/1.3 PlanFluor oil-immersion objective. Hydrogels were taken out of the 96 wells at least one day after formation and placed on 25-mm round coverslips. For hydrogels including live-cell imaging, hydrogels on coverslips were placed in a humidified, 37° C. chamber in 5% CO₂. For optogenetic activation a red laser diode was used (637 nm, 7.6 mW), or a scanning laser of NIR light (710 nm, 63 mW). For stimulation experiments, hydrogels were imaged before and after irradiation with mCherry channel.

### Exemplary Results and Discussion

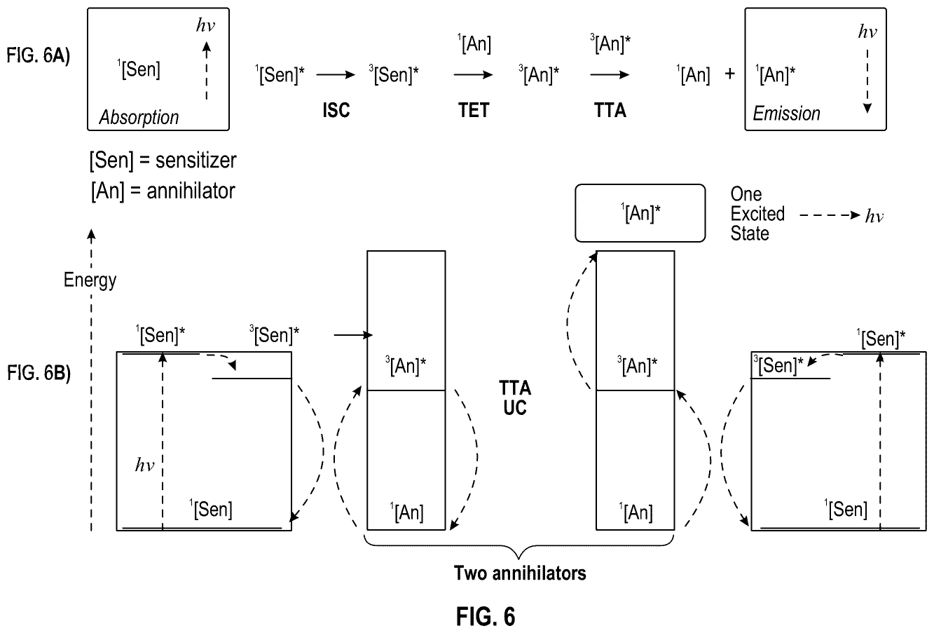

In order to develop photoactive biomaterials that operate by TTA-UC, it is important to consider the properties of the multiexciton systems together with the mechanism of upconversion, which has been extensively described in the literature^{25,26} (schematically detailed in FIG. 6). Previous studies have shown that TTA-UC hydrogels can be synthesized, but none have reported hydrogels containing cells dispersed within a 3D scaffold.⁵⁴ In a recent study, pluronic-based gels were simply used as a light source (NIR-to-blue-light) placed above 2D cultures of hippocampal neurons to trigger optogenetic responses. However, it is noted that similar optogenetic responses can be observed in 2D cultures by direct irradiation with blue light⁵. To obtain biocompatible, cell-laden TTA-UC hydrogels, it is possible to seek an integration the sensitizer/annihilator pairs within the core of oil-in-water emulsion droplets^{23,24,26,27} that could be incorporated into cell-laden hydrogel matrices (e.g., Fibrin gels). A number of requirements must be met so that the photoinduced processes can efficiently lead to the desired optogenetic response(s). First, the TTA-UC chromophores must operate with NIR light input (λ˜700 nm) and visible light output (λ<500 nm) under aerobic conditions, where oxygen is generally detrimental to TTA-UC chromophores. Second, the oil-in-water emulsion droplets must be biocompatible and must be robust within the 3D biomaterial composite, i.e., their contents must not be released into the environment. Finally, the biomaterials must contain NIR-silent/transparent components, but respond to the upconverted light within the hydrogel.

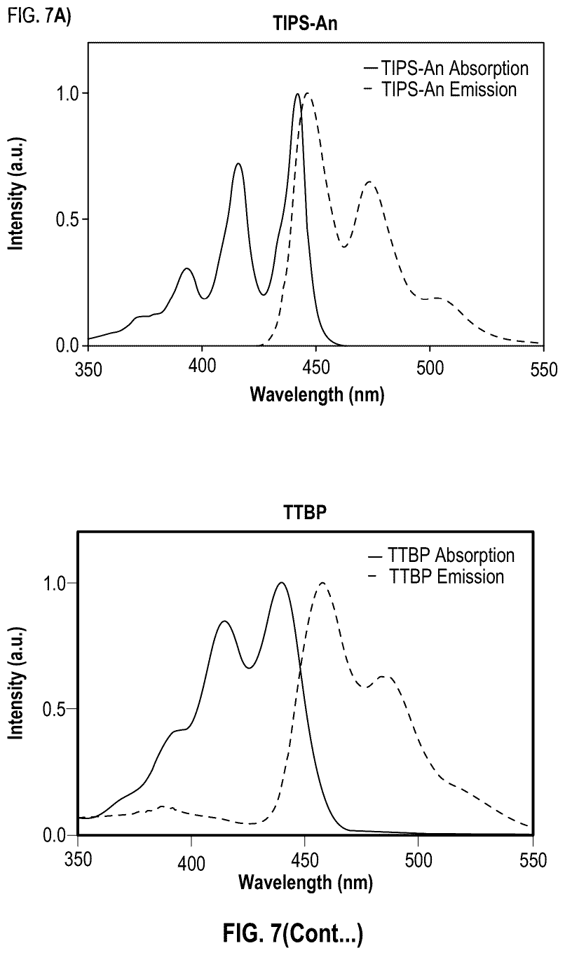

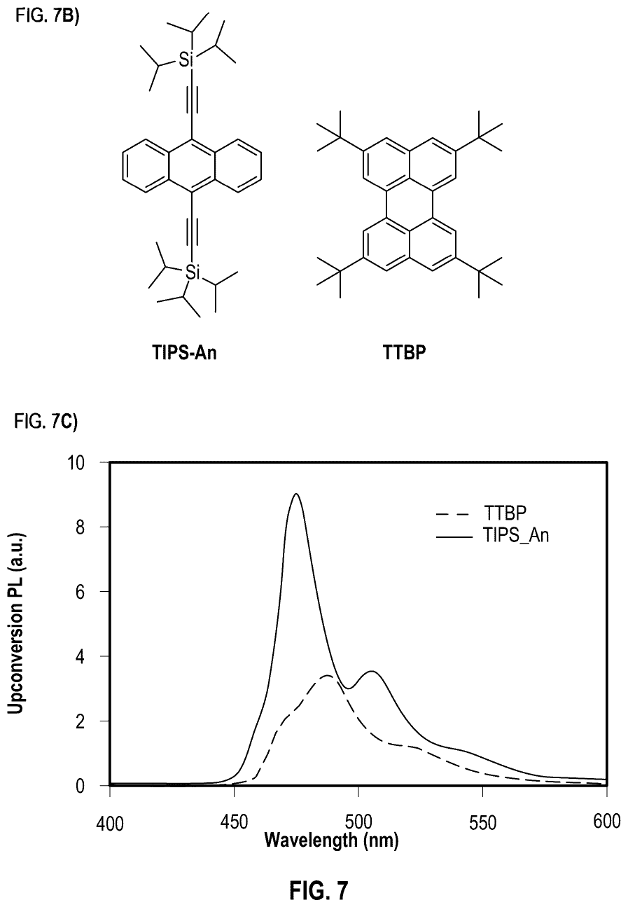

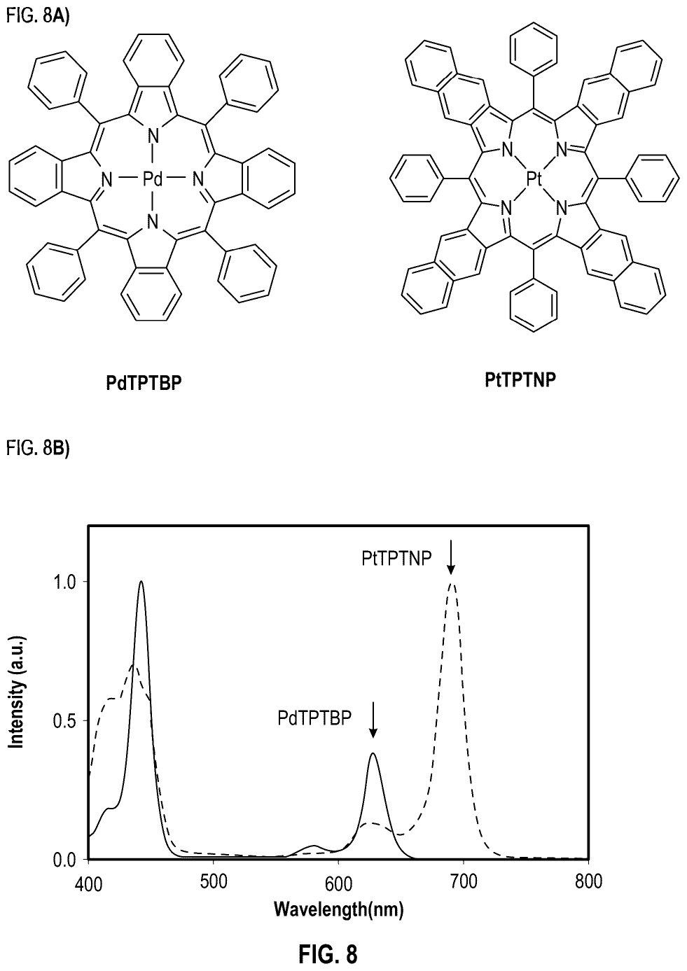

Considering the light input/output by TTA-UC to test an optogenetic response^{5,13}, it is noted that the majority of photoreceptors in engineered cells absorb blue light. In this study, Cry2olig, was used, which is activated by photons in the range of 400-500 nm (peaking at 450 nm)³⁰. Therefore, two chromophores can be recognized as efficient blue emitting annihilators: 9,10-((triisopropylsilyl)ethynyl)-anthracene³¹ (TIPS-An; see, e.g., FIGS. 1A0 to 1D)) and tetra-tert-butylperylene¹⁶ (TTBP; see, e.g., FIGS. 7A) and 7B)). These were paired with the triplet sensitizer palladium(II) meso-tetraphenyltetrabenzoporphyrin (PdTPTBP), which absorbs 640 nm light (Supplementary FIG. 8) emitted by red diodes, which are commercially available. While upconversion from red to blue light would give enhanced penetration of light, the need to efficiently sensitize within the biological transparency window would require longer wavelength of excitation (λ>700 nm). There are only a limited number of NIR-absorbing sensitizers³¹, but platinum(II) tetraphenyltetranaphthoporphyrin (PtTPTNP; FIGS. 1A), 1B) 8A and 8B)) can be paired with both TTBP¹⁶ and TIPS-An,³² exhibiting an apparent anti-Stokes shift ˜1.0 eV. While both annihilators work well, our studies focused on TIPS-An because it often outperforms TTBP in terms of upconversion efficiency, and it can be more readily prepared on a large scale.

Having identified suitable TTA-UC chromophores for red-to-blue and NIR-to-blue upconversion, it is possible to test the ability to achieve upconversion in biologically relevant systems. To date, most TTA-UC systems operate in deoxygenated organic solvents, which is antithetical to the environment that cultures of cells require.¹⁶ However, it is possible to encapsulate the chromophores within emulsions that contain a high boiling point non-polar organic solvent in the lipophilic core of the emulsion droplets²⁷ (see, e.g., FIGS. 1A) and 1B)). The organic solvents (i.e., 1,2,4-trichlorobenzene, toluene, and 1,3,5-trimethylbenzene [TMB]) are necessary to prevent aggregation of the TTA-UC chromophores, but they can be toxic to living organisms. However, it is possible to solubilize and improve the stability of TTA-UC chromophores using reductive oils^{33,34}. Therefore, a variety of emulsions for upconversion stability has been tested in the presence of oxygen and for cytotoxicity by varying the oil phase of the emulsions, including both natural soybean oil and D-limonene as antioxidant solvents and TMB as a control organic solvent. Finally, the cytotoxicity of three types of non-ionic surfactants has been tested (Pluronic F127, Tween 80 and Triton X-100).

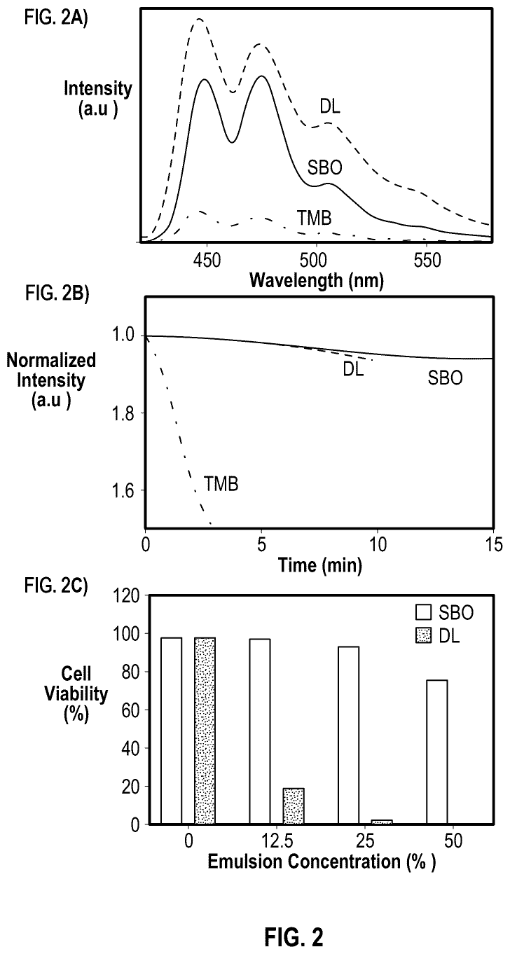

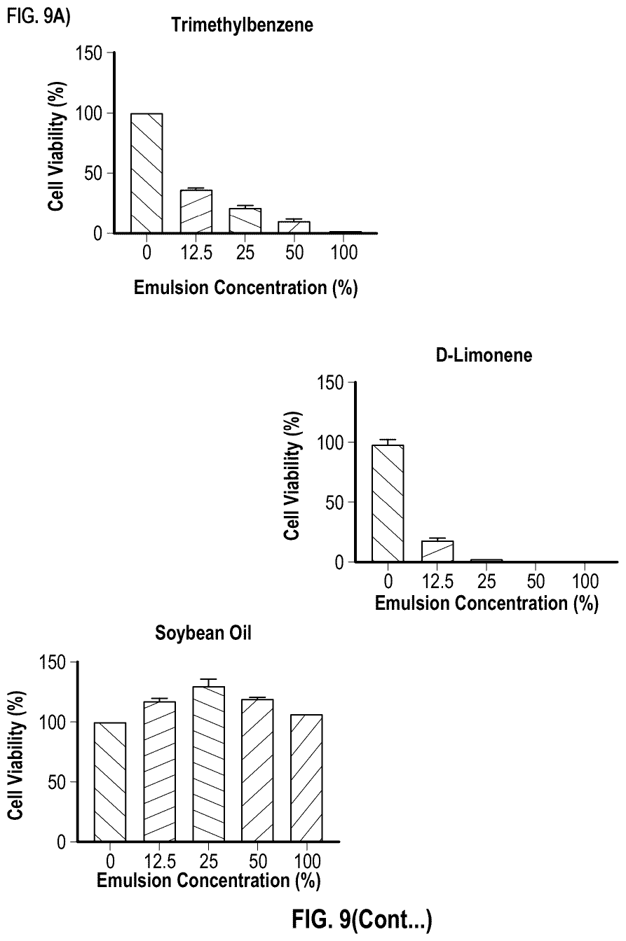

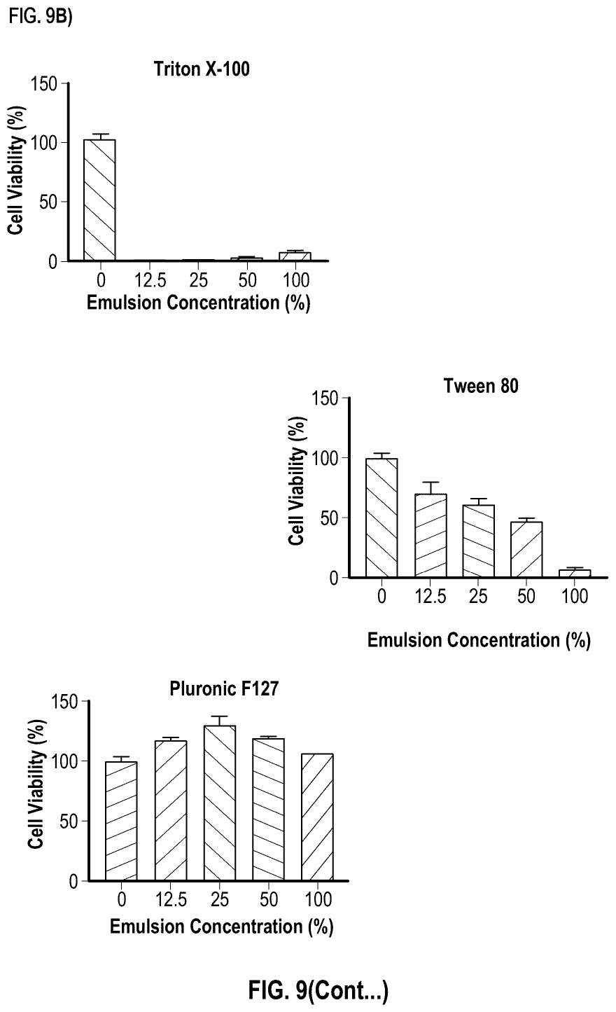

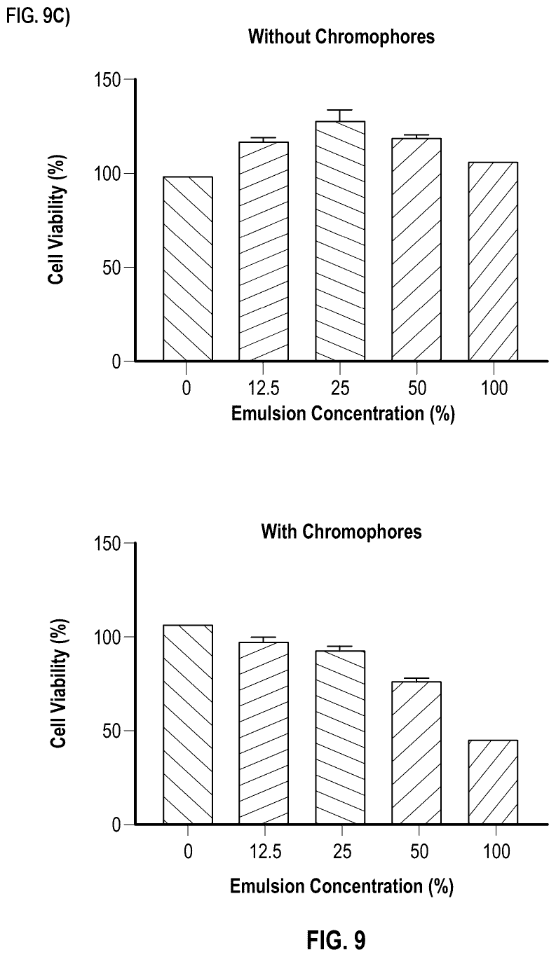

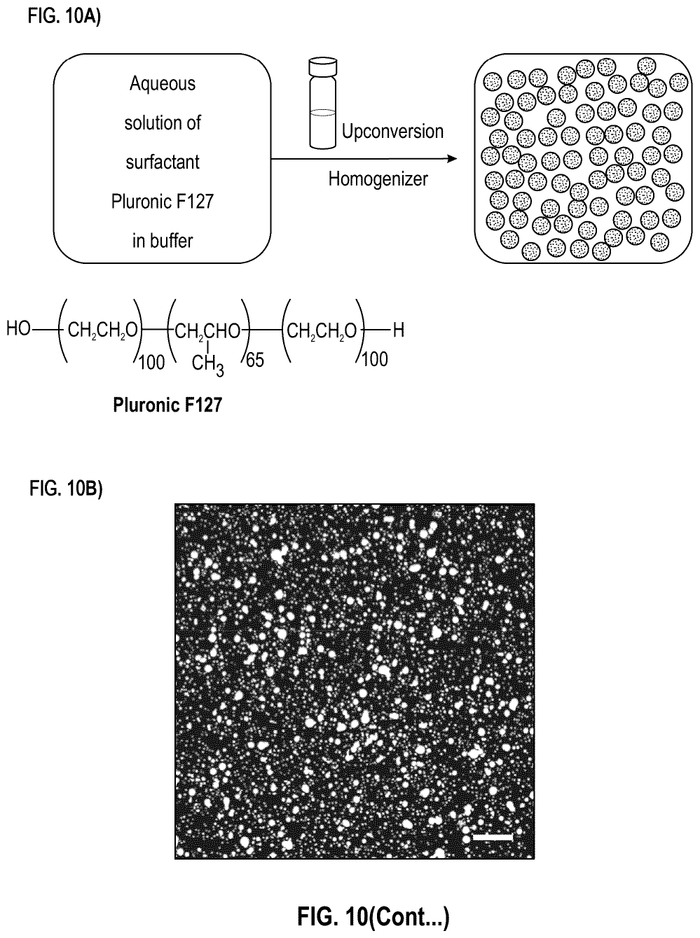

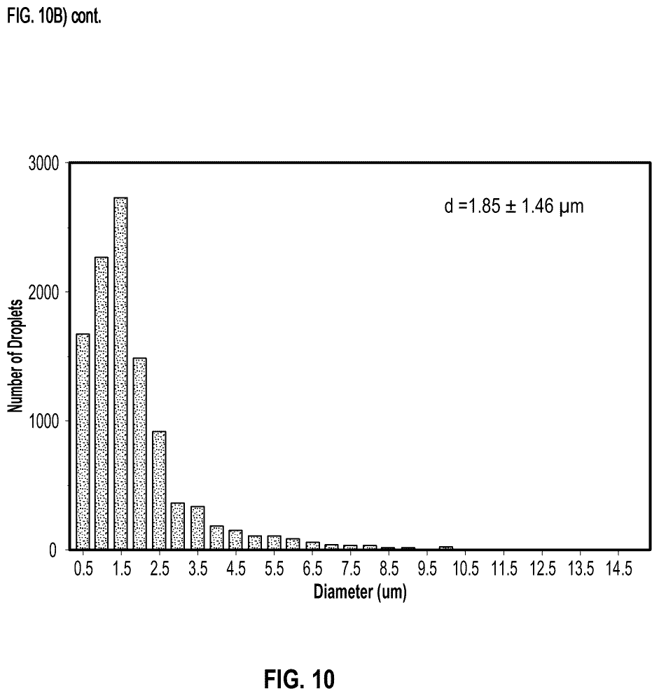

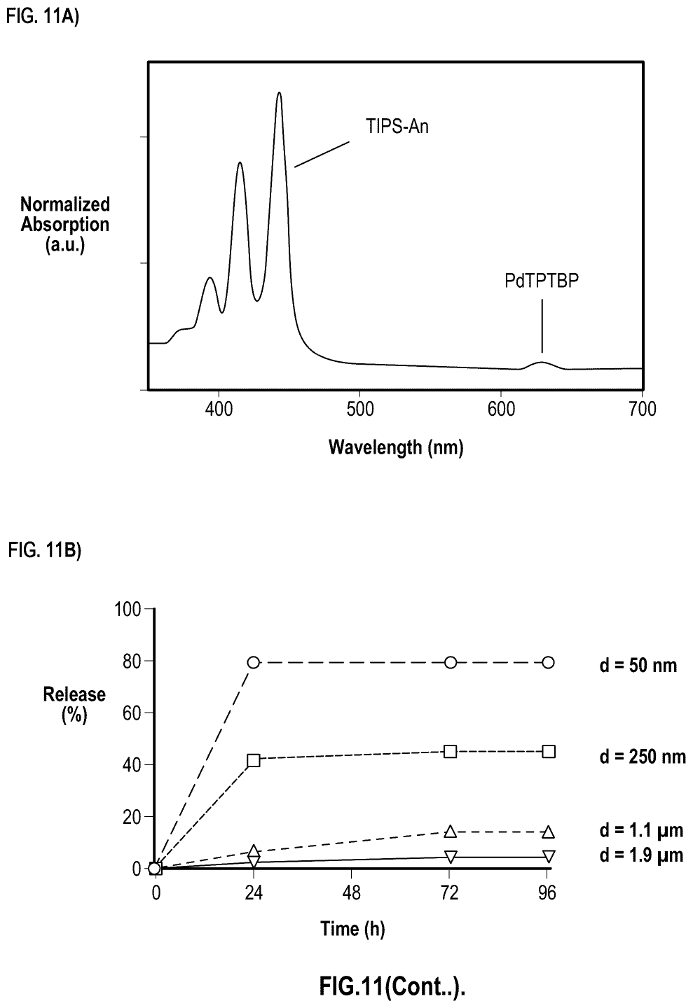

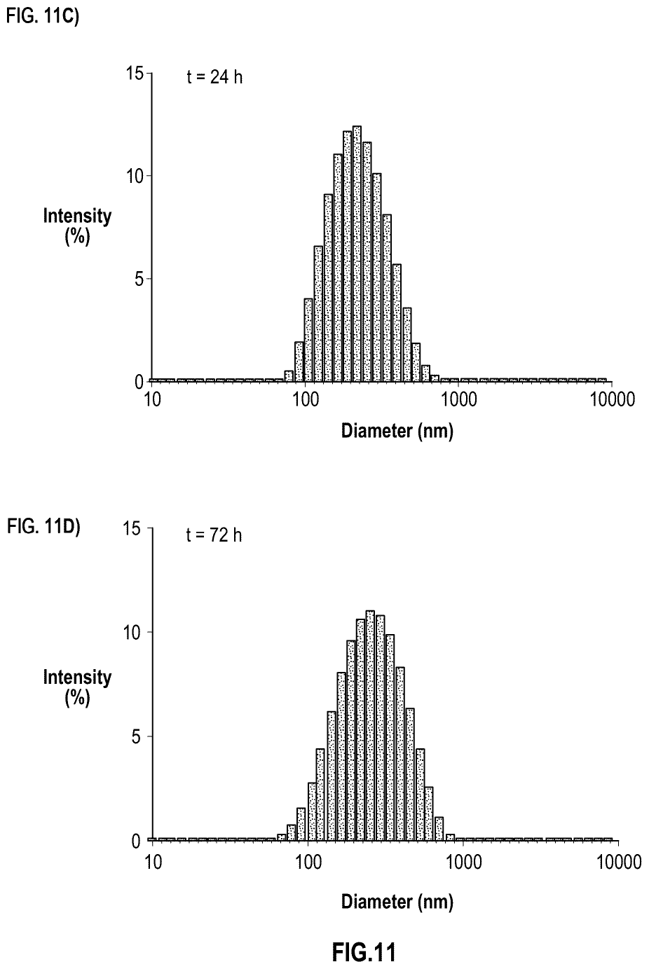

To evaluate the stability of the TTA-UC chromophores under irradiation in aerobic conditions, droplets based on Pluronic F127 surfactant has been used with the TIPS-An/PdTPTBP annihilator/sensitizer pair. The emulsions were exposed to continuous irradiation with a 637 nm laser for 15 minutes (76 mW/mm²) and a spectral detector was used to measure luminescence of visible light at one-minute intervals (spectral range 400-580 nm). FIG. 2A) shows the photoluminescence emission spectrum of TIPS-An from the TTA-UC emulsions. it was found that both soybean oil and D-limonene as the oil phase of the emulsion qualitatively exhibited higher upconversion intensity than TMB (FIG. 2A) and the luminescence remained constant for the entire duration of the experiment (15 minutes: FIG. 2B). On the other hand, the luminescence of droplets containing TMB was further reduced to 50% of its initial luminescence within 4 minutes of irradiation (see, e.g., FIG. 2B)). Since both soybean oil and D-limonene provided better stability to the chromophores than TMB, the biocompatibility of these emulsions can be examined. Upon exposing cells to the upconversion emulsions for one hour using Pluronic F127 as the surfactant, with and without chromophores and at different concentrations, the D-limonene emulsion droplets were found to be toxic to the cells. Interestingly, cells exposed to low concentrations of D-limonene droplets without chromophores had low viability, while the soybean oil droplets were found to be biocompatible, with and without chromophores, at high concentrations (see, e.g., FIG. 2C) and FIGS. 9A) and 9B)). Among the three surfactants that were tested (Pluronic F127, Tween 80 and Triton X-100), only Pluronic F127 was found to be biocompatible and nontoxic to the cells at high concentrations (see, e.g., FIG. 9B)). Thus, it was concluded that using soybean oil in the core of the droplets and Pluronic F127 (10% wt) as a surfactant were biocompatible and resulted in efficient upconversion in aqueous and air saturated environment. In our studies, it was determined that the size of the emulsion droplets was important. While other work has focused on nanoscale micelles²⁷, it was found that increasing the size of the emulsion droplets is necessary to prevent their release from the hydrogel matrix into the environment (see, e.g., FIGS. 10A) to 11). Thus, in the course of this review, microscale droplets with a mean diameter of ˜1.9 μm have been used to entrap them within the hydrogel matrix without leaching.

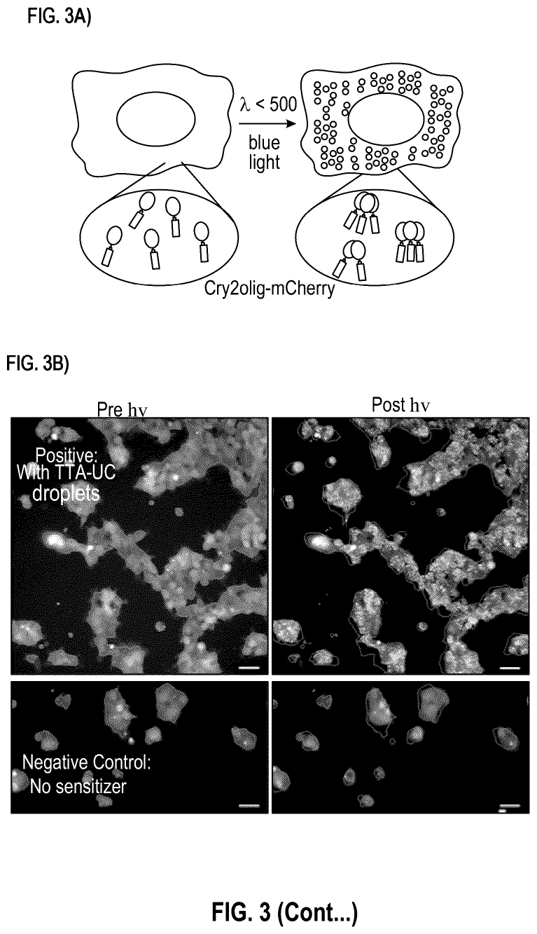

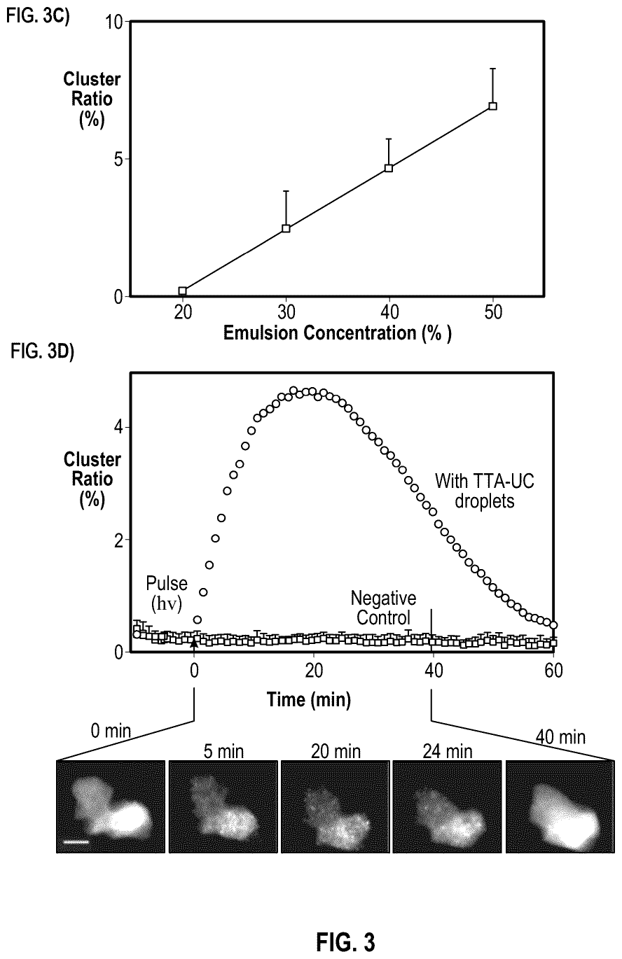

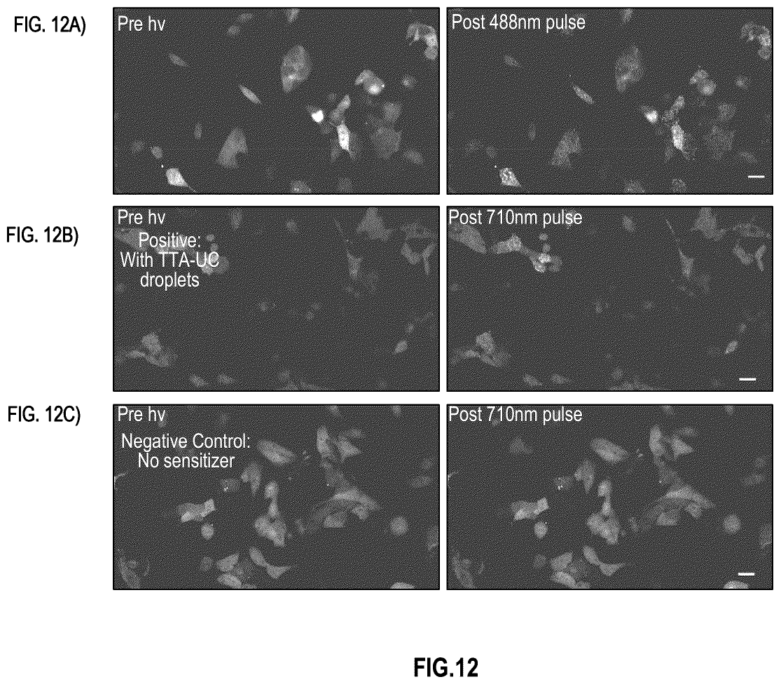

Having identified appropriate non-cytotoxic TTA-UC emulsions, it was tested whether the energy output of the TTA-UC process from the emulsions would suffice to activate photoreceptors in optogenetic cells in 2D cell cultures³⁰, as a first step before introducing the hydrogel matrix. As a model system, e.g., the Arabidopsis photoreceptor Cry2olig was utilized, which induces rapid and reversible protein oligomerization in response to blue light (FIG. 3A)^{28,29}. For the 2D experiments, HeLa cells were engineered to stably express Cry2 fused to mChery construct (Cry2olig-mChy) and were cultured as monolayer on glass-bottom plates one day prior to photostimulation. A light emitting diode (LED) with a pulse of blue light (λₑₓ=488 nm, 1 sec pulse) was used, and such exemplary setup confirmed the formation of clusters of Cry2oligo in response to blue light (see, e.g., FIG. 12A)). Next, the TTA-UC emulsion (TIPS-An:PdTPTBP) or control emulsion (TIPS-An only) were mixed into the cell media at various concentrations (10 to 50% volume). For upconversion-mediated excitation, the cells were irradiated with a pulse of red light (λₑₓ=637 nm, 1 sec, 11 to 55 mW). Fluorescent microscopy imaging was used to track the formation of clusters of the Cry2oligo-mChy construct before irradiation and after irradiation. Before stimulation, most of the Cry2oligo-mChy signal appeared to be distributed throughout the cell cytoplasm (see, e.g., FIG. 3B), pre-hv). Upon red light stimulation under aerobic conditions in the presence of the upconversion emulsion, a cluster formation was observed of Cry2oligo-mChy proteins (see, e.g., FIG. 3B) top, post-hv). A linear correlation was found between the concentration of the upconversion emulations in the cell media and the number of clusters formed, with a threshold of 20% volume for cluster formation (FIG. 3C). The control experiment without the inclusion of the light-absorbing sensitizer in the emulsion, showed the lack of cluster formation (see, e.g., FIG. 3B), negative control, post-hv). Furthermore, FIG. 3D shows the reversibility of the upconversion-mediated optogenetic process, as the number of clusters increase after the light pulse and then decreased overtime, as expected from Cry2oligo-mChy construct²⁸. Of note, cells that were exposed to the TTA-UC emulsions under irradiation remained vital for the duration of the experiment (70 min) for all TTA-UC emulsion concentrations tested. Next, similar experiments were carried out, but using the NIR-to-blue upconversion emulsion loaded with PtTPTNP as the sensitizer (TIPS-An:PtTPTNP, λₑₓ=710 nm). Not surprisingly, similar upconversion-mediated clustering in response to NIR light stimulation was observed (see, e.g., FIG. 12B), while no clustering was detected with control emulsions (TIPS-An only; FIG. 12C).

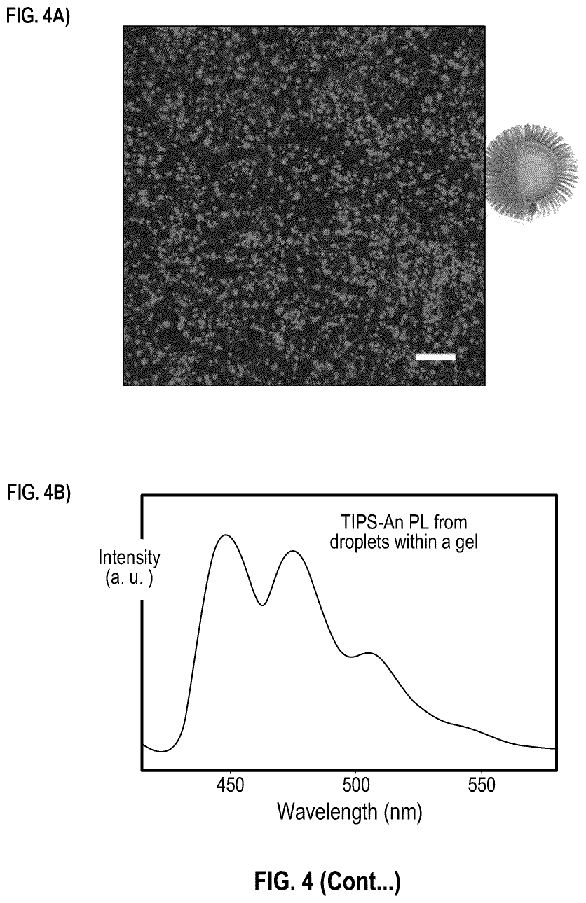

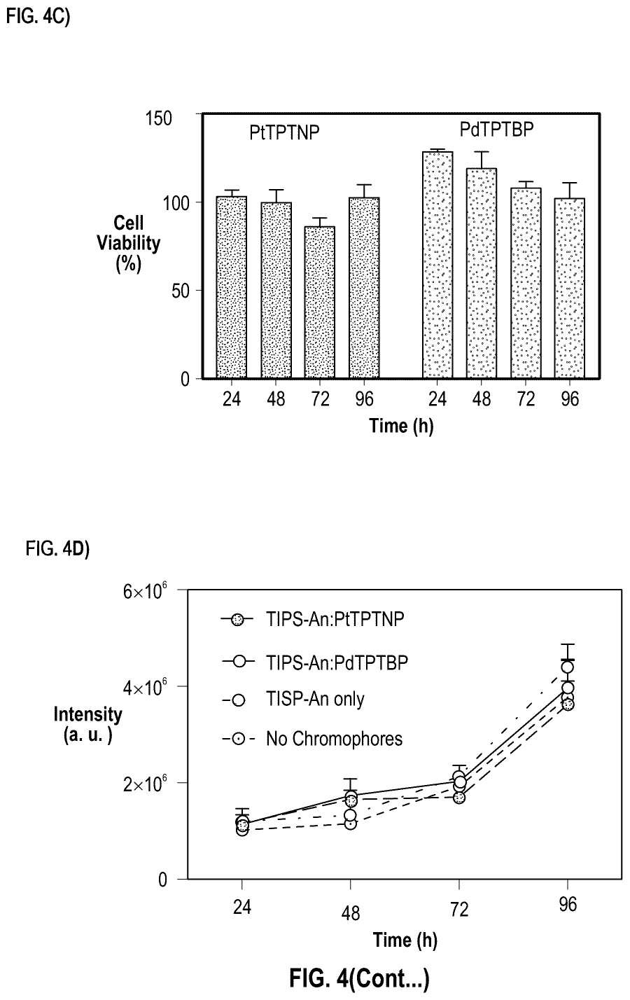

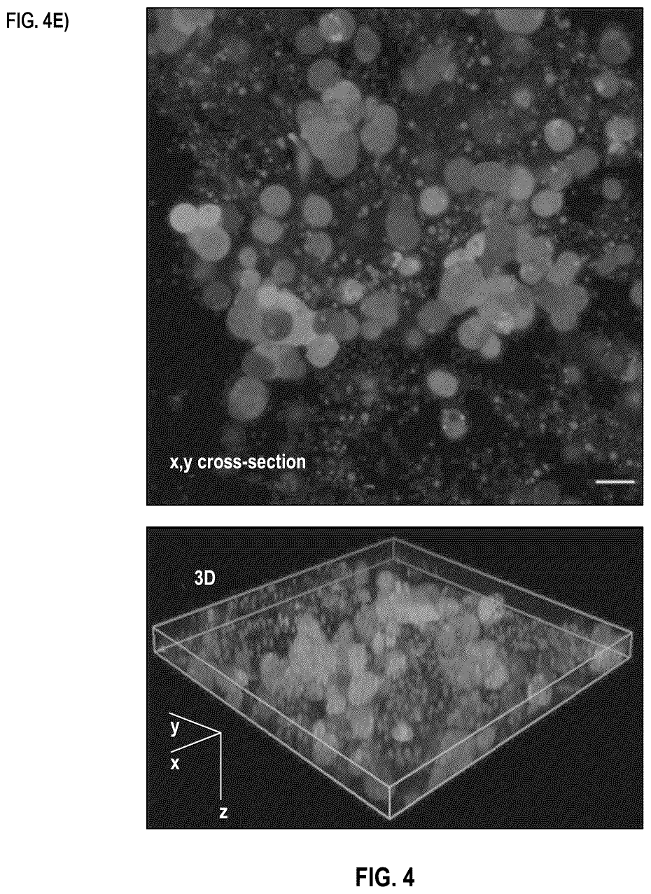

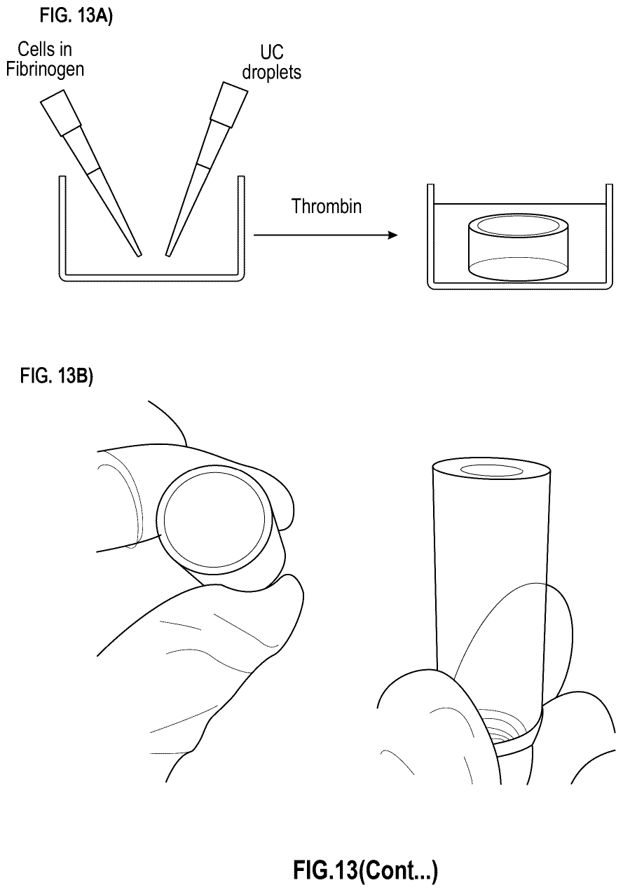

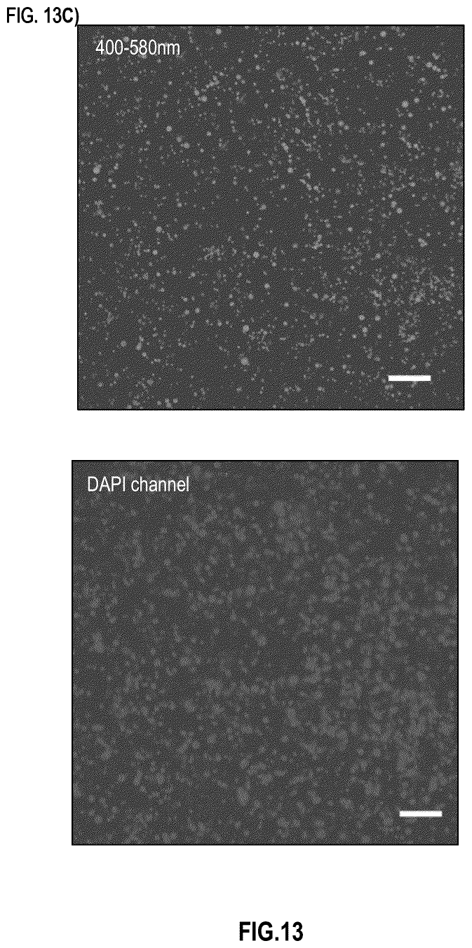

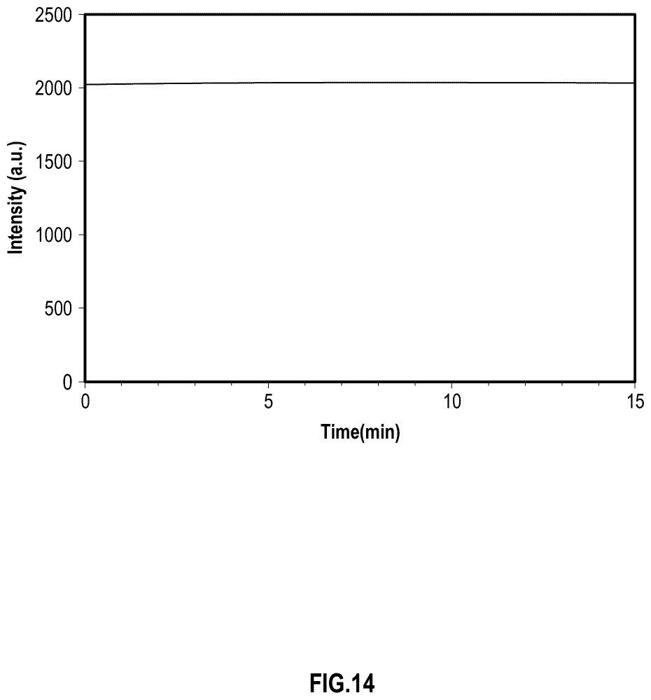

Encouraged that the TTA-UC emulsions triggered an optogenetic response in a monolayer of cells (2D), it was sought to test the performance of the upconversion emulsion droplets dispersed within a fibrin gel (3D), a commonly used hydrogel matrix³⁵. The cell-loaded TA-UC hydrogels were prepared by adding the TTA-UC emulsion (5% by volume) to the cell-containing fibrinogen solution before the biopolymer was cross-linked with the Thrombin enzyme (FIG. 13A). From the resulting free-standing gel, blue light emission can be observed with the naked eye upon stimulation with red light (see, e.g., FIG. 1C) and FIG. 13B)). Moreover, FIG. 4A shows the confocal microscopy images of the emitted light from the widely dispersed droplets within TTA-UC hydrogels based on TIPS-An:PdTPTBP (see, e.g., FIG. 13C) for other detection windows). From these gels, the PL emission spectrum shown in FIG. 4B matches that of TIPS-An. The stability of the TTA-UC signal in the hydrogels (TIPS-An:PdTPTBP) was tested by continuous irradiation from a red laser (λₑₓ=637 nm, 76 mW/mm²), and no loss of signal was observed after 15 min (see, e.g., FIG. 14), similar to the signal stability in 2D (see, e.g., FIG. 2B)). The 3D resolution of the TTA-UC hydrogels is fundamentally defined by the penetration depth of red (λ˜640 nm) and NIR (λ>700 nm) light through the cell-laden hydrogels. It is well known that the scattering and absorption coefficients of biological tissues are strongly wavelength dependent.^{[39]}

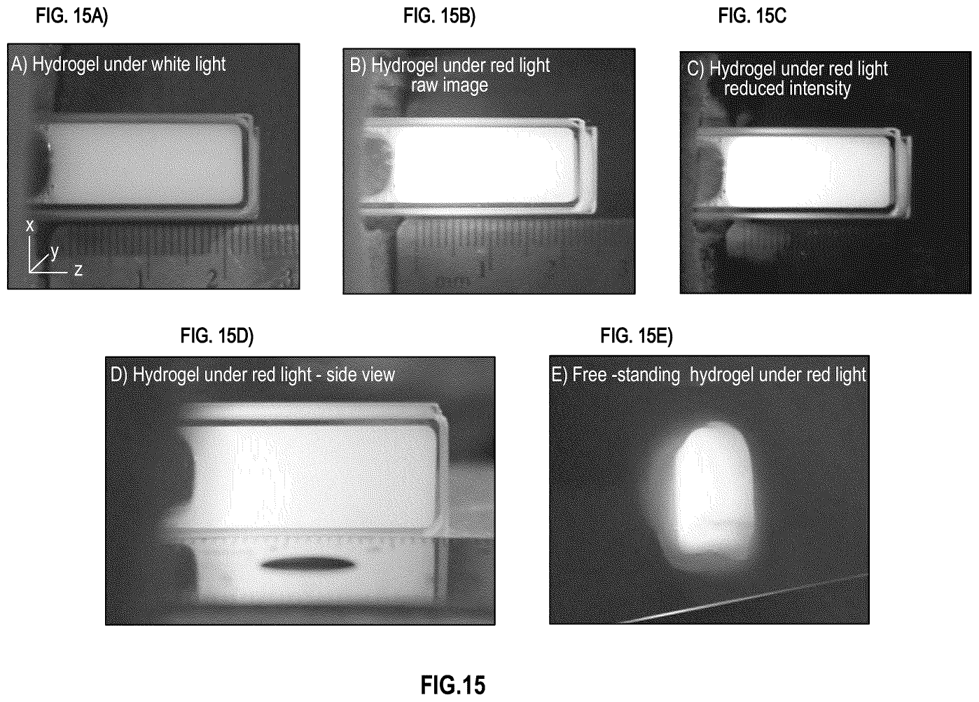

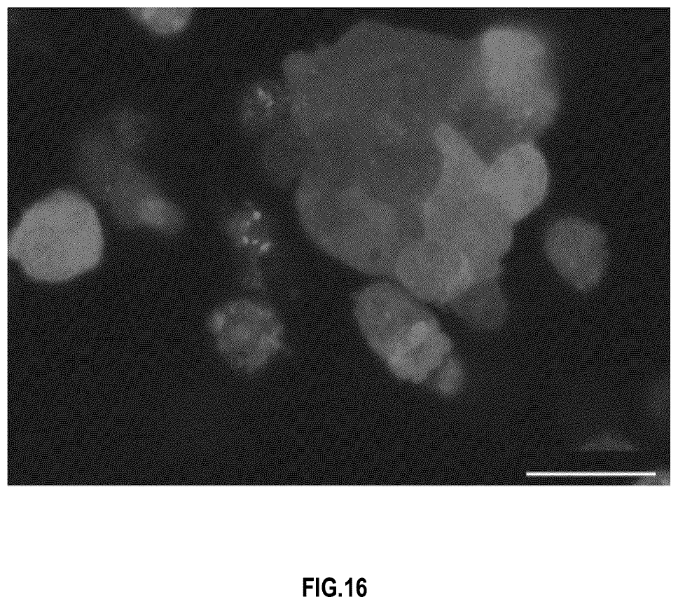

While blue light offers limited penetration through tissue, the advantage of using red and NIR light for excitation of a system is that these wavelengths lie within the biological optical window (640-950 nm). For example, for light penetration through human skin, red and NIR light is extinguished ˜5 mm beneath the surface of the skin, whereas blue light penetrates ˜1 mm into tissue.^{[20]} Given that the hydrogels are opaque and scatter blue light, the penetration of 640 nm light through the upconverting hydrogel was qualitatively characterized. A sample with dimensions of 1 cm×1 cm×2.5 cm was synthesized in a cuvette and irradiated through the long axis (see, e.g., FIG. 15) at ambient conditions. In this sample, upconverted blue light was visible and photographs show scattered blue light emanating throughout the opaque gel. Next, cell viability assays show that the hydrogels are biocompatible for at least 4 days post-encapsulation using both TIPS-An:PdTPTBP and TIPS-An:PtTPTNP emulsion droplets within the hydrogel (see, e.g., FIG. 4C)). Moreover, the hydrogel supports cell proliferation as shown by the increase in signal over time (see, e.g., FIG. 4D)). Furthermore, microscopy images show the coexistence of the cells and emulsion droplets dispersed within the upconversion hydrogel for at least four days (see, e.g., FIG. 4E) and FIG. 16).

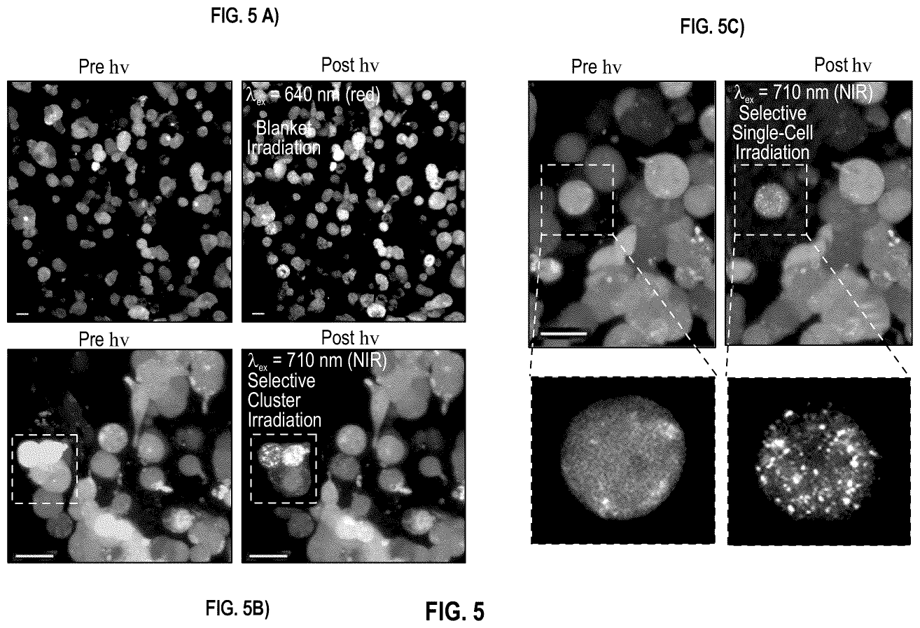

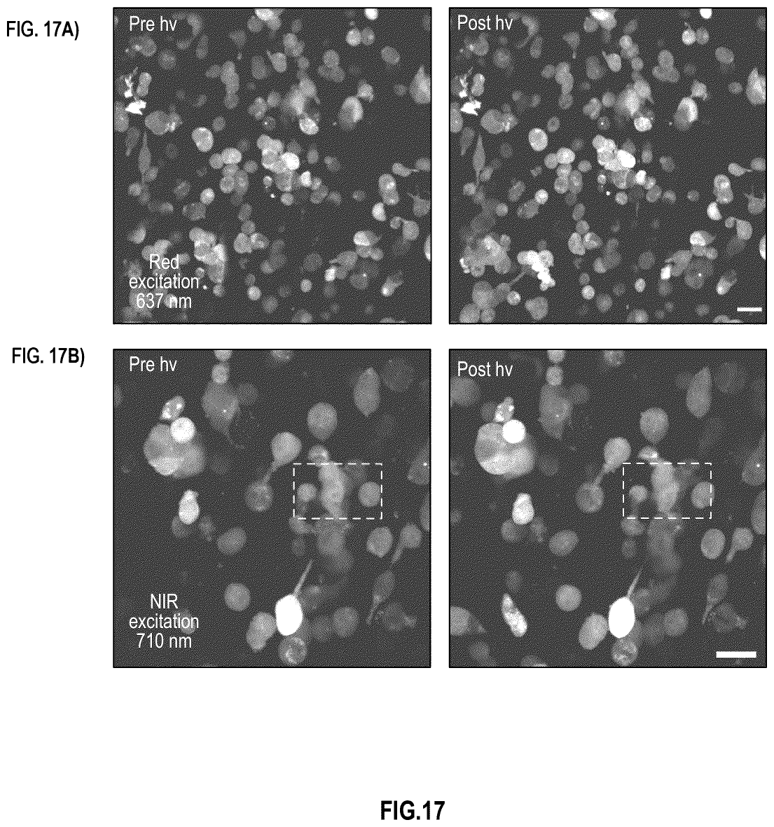

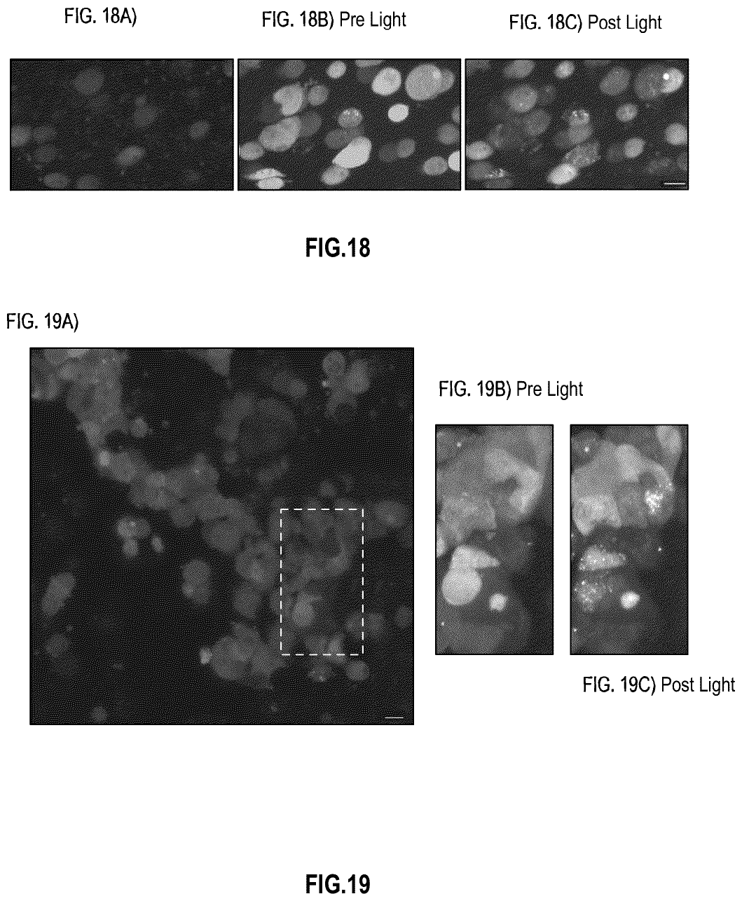

In order to test the ability to stimulate the optogenetic cells inside the cell-laden TTA-UC biomaterial using red and NIR light, according to exemplary embodiments of the present disclosure, the 3D optogenetic response from the engineered HeLa cells were investigated. First, HeLa cells expressing Cry2oliog-mChy were encapsulated into the fibrin hydrogels containing 5% (by volume) of the TTA-UC emulsion (TIPS-An:PdTPTBP or TIPS-An:PtTPTNP) or control emulsions (TIPS-An only). Two days post encapsulation in the hydrogel, the cells were stimulated with a red or NIR laser and imaged by confocal microscopy. As shown in FIG. 5A), upon blanket irradiation with red-light (λₑₓ=637 nm, 7.6 mW), according to exemplary embodiments of the present disclosure, the formation of Cry2oligo clusters have been clearly observed—the primary optogenetic response, while the control hydrogels without the sensitizer did not show clustering (see. e.g., FIG. 17)). The exemplary embodiments of the present disclosure were able to observe the formation of clusters upon stimulating the cell-laden TTA-UC gels with a NIR scanning laser (λₑₓ=710 nm, 63 mW)(FIG. 18).

Given the homogenous optogenetic response of the cells in 3D, it was hypothesized that local irradiation could illicit an optogenetic response from a select group of cells. The most widely studied methods to trigger/control cellular processes can only be done at steady state through chemical cues in the culture media^{[40,41]} or by modifications to their immediate environment in 2D^{[42-46]} and 3D.^{[47]} Thus, it is of utmost interest to develop technologies to achieve spatiotemporal control of cellular behavior, down to single-cell resolution.⁴⁹ By focusing the microscope NIR laser on a specific group of cells, according to an exemplary embodiment of the present disclosure, it was possible to trigger the TTA-UC process in their vicinity with precision, which then led to the clustering of Cry2olig. FIG. 5B shows confocal images of the cells dispersed in the 3D matrix before focused irradiation (pre hv) and after NIR light exposure only in specific sites of irradiation (post hv, the irradiated region is highlighted in the images). It was remarkable to observe the lack of Cry2olig cluster formation from cells within the vicinity of focused irradiation of the TTA-UC droplets. According to an exemplary embodiment of the present disclosure, using long-wavelength light can trigger cellular processes with spatiotemporal control in 3D. Moreover, it is possible to trigger the optogenetic response of a single cell (FIG. 5C). To date, such level of specificity in a 3D matrix is unprecedented. This class of biomaterials can open up new avenues of exploration of cell signaling mechanisms, 3D printing, drug delivery, and tissue engineering, among others.^{50,51} It is noteworthy that these experiments were reproducible over several days and it were able to possible to image and stimulate cells in the hydrogels on days 0-3 post encapsulation (see FIG. 18 and FIG. 19). The control hydrogels without the sensitizer did not show clustering of the proteins within a region on interest under NIR light irradiation (FIG. 16B).

According to certain exemplary embodiments of the present disclosure, it is possible to employ the unconventional photophysical light-energy harvesting process of TTA-UC to manipulate cell behavior with spatiotemporal control. In exemplary embodiments of the present disclosure, provided is a class of 3D biomaterials capable of absorbing red/NIR light, which is upconverted to blue light by TTA-UC. In exemplary embodiments of the present disclosure, photon upconversion hydrogel biomaterials can be provided, which are suitable to photochemically induce optogenetic responses in cell-laden hydrogels enabled by TTA-UC. With these systems, it is possible to photostimulate cells within a 3D biomaterial, overcoming the limitations imposed by direct irradiation with blue light. Adapting upconversion chromophores to function under ambient conditions has been an obstacle for interfacing these systems with living organisms as most systems are limited to deoxygenated organic solvents. In addition, most TTA-UC reported systems are limited to visible to visible upconversion, however for biological applications NIR to blue upconversion is highly desired²⁵. The exemplary embodiments of the present disclosure were able to overcome these challenges by incorporating the upconversion chromophores into emulsions containing soybean oil, a reductive solvent that consumes any generated singlet oxygen by TTA-UC, thus increasing the photostability without compromising biocompatibility and cytotoxicity. Furthermore, the rather large microdroplets of the emulsion prevented leaching from the hydrogel to the environment. Overall, the composition of the 3D TTA-UC biomaterials according to certain exemplary embodiments of the present disclosure led to the photochemical activation of optogenetic HeLa cells, within 3D biomaterials. Moreover, the exemplary embodiments of the present disclosure were able to achieve the primary optogenetic response of a single-cell within a cell-laden hydrogel, exerting spatial temporal control using a scanning laser. The successful development of 3D biomaterials is especially significant because the methodology introduces a new way to initiate photochemical processes of optogenetically engineered cells using low-energy light, with unprecedented precision.

The foregoing merely illustrates the principles of the disclosure. Various modifications and alterations to the described embodiments can be apparent to those skilled in the art in view of the teachings herein. It can thus be appreciated that those skilled in the art can be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as can be understood by those having ordinary skill in the art. In addition, certain terms used in the present disclosure, including the specification, drawings and claims thereof, can be used synonymously in certain instances, including, but not limited to, for example, data and information. It can be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly incorporated herein in its entirety.

Various references relating to the present disclosure are listed below, the entire disclosures of which references are hereby incorporated by reference.

### REFERENCES

The following references are hereby incorporated by reference in their entireties:
S-1. Huang K, Dou Q, Loh X J. Nanomaterial mediated optogenetics: opportunities and challenges. RSC Adv. 2016 June; 6: pp. 60896-906.
S-2. Ferenczi E A, Tan X, Huang C L. Principles of Optogenetic Methods and Their Application to Cardiac Experimental Systems. Front Physiol. 2019 September; 10: p. 1096.
S-3. Shabahang S, Kim S, Yun S H. Light-Guiding Biomaterials for Biomedical Applications. Adv Funct Mater. 2018 June; 28(24).
S-4. Huang L, Kakadiaris E, Vaneckova T, Huang K, Vaculovicova M, Han G. Designing next generation of photon upconversion: Recent advances in organic triplet-triplet annihilation upconversion nanoparticles. Biomaterials. 2019 February; 10: pp. 77-86.
(1) Lutolf, M. P.; Hubbell, J. A. Synthetic Biomaterials as Instructive Extracellular Microenvironments for Morphogenesis in Tissue Engineering. Nature Biotechnology, 2005, 23, 47-55.
(2) Repina, N. A.; Rosenbloom, A.; Mukherjee, A.; Schaffer, D. V.; Kane, R. S. At Light Speed: Advances in Optogenetic Systems for Regulating Cell Signaling and Behavior. Annu. Rev. Chem. Biomol. Eng. 2017, 8, 13-39.
(3) Huang, K.; Dou, Q.; Loh, X. J. Nanomaterial Mediated Optogenetics: Opportunities and Challenges. RSC Adv. 2016, 6, 60896-60906.
(4) Ash, C.; Dubec, M.; Donne, K.; Bashford, T. Effect of Wavelength and Beam Width on Penetration in Light-Tissue Interaction Using Computational Methods. Lasers Med. Sci. 2017, 32, 1909-1918.
(5) Stolik, S.; Delgado, J. A.; Pérez, A.; Anasagasti, L. Measurement of the Penetration Depths of Red and near Infrared Light in Human “ex Vivo” Tissues. J. Photochem. Photobiol. B Biol. 2000, 57, 90-93.
(6) Askes, S. H. C.; Bonnet, S. Solving the Oxygen Sensitivity of Sensitized Photon Upconversion in Life Science Applications. Nature Reviews Chemistry, 2018, 2, 437-452.
(7) Hussey, G. S.; Dziki, J. L.; Badylak, S. F. Extracellular Matrix-Based Materials for Regenerative Medicine. Nat. Rev. Mater. 2018, 3, 159-173.
(8) Pun, A. B.; Campos, L. M.; Congreve, D. N. Tunable Emission from Triplet Fusion Upconversion in Diketopyrrolopyrroles. J. Am. Chem. Soc. 2019, 141, 3777-3781.
(9) Pun, A. B.; Sanders, S. N.; Sfeir, M. Y.; Campos, L. M.; Congreve, D. N. Annihilator Dimers Enhance Triplet Fusion Upconversion. Chem. Sci. 2019, 10, 3969-3975.
(10) Ravetz, B. D.; Pun, A. B.; Churchill, E. M.; Congreve, D. N.; Rovis, T.; Campos, L. M. Photoredox Catalysis Using Infrared Light via Triplet Fusion Upconversion. Nature, 2019, 565, 343-346.
(11) Tao, Y.; Huang, A. J. Y.; Hashimotodani, Y.; Kano, M.; All, A. H.; Tsutsui-kimura, I.; Tanaka, K. F.; Liu, X.; Mchugh, T. J. Near-infrared Deep Brain Stimulation via Upconversion Nanoparticle-Mediated Optogenetics.Pdf. Science (80-.). 2018, 684, 679-484.
(12) Goglia, A. G.; Toeitcher, J. E. A Bright Future: Optogenetics to Dissect the Spatiotemporal Control of Cell Behavior. Curr. Opin. Chem. Biol. 2019, 48, 106-113.
(13) Zhou, J.; Liu, Q.; Feng, W.; Sun, Y.; Li, F. Upconversion Luminescent Materials: Advances and Applications. Chem. Rev. 2015, 115, 395-465.
(14) Tibbitt, M. W.; Anseth, K. S. Hydrogels as Extracellular Matrix Mimics for 3D Cell Culture. Biotechnol. Bioeng. 2009, 103, 655-663.
(15) DeForest, C. A.; Anseth, K. S. Advances in Bioactive Hydrogels to Probe and Direct Cell Fate. Annu. Rev. Chem. Biomol. Eng. 2012, 3, 421-444.
(16) Fleischer, S.; Shapira, A.; Feiner, R.; Dvir, T. Modular Assembly of Thick Multifunctional Cardiac Patches. Proc. Natl. Acad. Sci. 2017, 114, 1898-1903.
(17) Lian, X.; Hsiao, C.; Wilson, G.; Zhu, K.; Hazeltine, L. B.; Azarin, S. M.; Raval, K. K.; Zhang, J.; Kamp, T. J.; Palecek, S. P. Robust Cardiomyocyte Differentiation from Human Pluripotent Stem Cells via Temporal Modulation of Canonical Wnt Signaling. Proc. Natl. Acad. Sci. U.S.A. 2012, 109.
(18) Burridge, P. W.; Matsa, E.; Shukla, P.; Un, Z. C.; Churko, J. M.; Ebert, A. D.; Lan, F.; Diecke, S.; Huber, B.; Mordwinkin, N. M.; Plews, J. R.; Abilez, O. J.; Cui, B.; Gold, J. D.; Wu, J. C. Chemically Defined Generation of Human Cardiomyocytes. Nat. Methods 2014, 11, 855-860.
(19) Pun, A. B.; Asadpoordarvish, A.; Kumarasamy, E.; Tayebjee, M. J. Y.; Niesner, D.; McCamey, D. R.; Sanders, S. N.; Campos, L, M.; Sfeir, M. Y. Ultra-Fast Intramolecular Singlet Fission to Persistent Multiexcitons by Molecular Design. Nat. Chem. 2019, 11, 821-828.
(20) Busby, E.; Xia, J.; Wu, Q.; Low, J. Z.; Song, R.; Miller, J. R.; Zhu, X. Y.: Campos, L. M.; Sfeir, M. Y. A Design Strategy for Intramolecular Singlet Fission Mediated by Charge-Transfer States in Donor-Acceptor Organic Materials. Nat. Mater. 2015, 14, 426-433.
(21) Tayebjee, M. J. Y.; Sanders, S. N.; Kumarasamy, E.; Campos, L. M.; Sfeir, M. Y.; McCamey, D. R. Quintet Multiexciton Dynamics in Singlet Fission. Nat. Phys. 2017, 13, 182-188.
(22) Cimetta, E.; Cannizzaro, C.; James, R.; Biechele, T.; Moon, R. T.; Elvassore, N.; Vunjak-Novakovic, G. Microfluidic Device Generating Stable Concentration Gradients for Long Term Cell Culture: Application to Wnt3a Regulation of β-Catenin Signaling. Lab Chip 2010, 10, 3277-3283.
(23) Ronaldson-Bouchard, K.; Ma, S. P.; Yeager, K.; Chen, T.; Song, L. J.; Sirabella, D.; Morikawa, K.; Teles, D.; Yazawa, M.; Vunjak-Novakovic, G. Advanced Maturation of Human Cardiac Tissue Grown from Pluripotent Stem Cells. Nature 2018, 556, 239-243.
(24) Wan, L. Q.; Ronaldson, K.; Park. M.; Taylor, G.; Zhang, Y.; Gimble, J. M.; Vunjak-Novakovic, G. Micropatterned Mammalian Cells Exhibit Phenotype-Specific Left-Right Asymmetry. Proc. Natl. Acad. Sci. U.S.A. 2011, 108, 12295-12300.
(25) Vuniak-Novakovic, G.; Scadden, D. T. Biomimetic Platforms for Human Stem Cell Research. Cell Stem Cell, 2011, 8, 252-261.
(26) Li, Y.: Meng. H.; Liu, Y.; Lee, B. P. Fibrin Gel as an Injectable Biodegradable Scaffold and Cell Carrier for Tissue Engineering. Scientific World Journal, 2015, 2015, 1-10.
(27) Repina, N. A.; Bao, X.; Zimmermann, J. A.; Joy, D. A.; Kane, R. S.; Schaffer, D. V. Optogenetic Control of Wnt Signaling for Modeling Early Embryogenic Patterning with Human Pluripotent Stem Cells. bioRxiv 2019, 665695.
(28) Sanders, S. N.; Gangishetty, M. K.; Sfeir, M. Y.: Congreve, D. N. Photon Upconversion in Aqueous Nanodroplets. J. Am. Chem. Soc. 2019, 141, 9180-9184.
(29) Gray, V.; Dzebo, D.; Lundin, A.; Alborzpour, J.; Abrahamsson, M.; Albinsson, B.; Moth-Poulsen, K. Photophysical Characterization of the 9,10-Disubstituted Anthracene Chromophore and Its Applications in Triplet-Triplet Annihilation Photon Upconversion. J. Mater. Chem. C2015, 3, 11111-11121.
(30) Liu, Q.; Xu, M.; Yang, T.; Tian, B.; Zhang, X.; Li, F. Highly Photostable Near-IR-Excitation Upconversion Nanocapsules Based on Triplet-Triplet Annihilation for in Vivo Bioimaging Application. ACS Appl. Mater. Interfaces 2018, 10, 9883-9888.
(31) Kumar, A.; Zo, S. M.; Kim, J. H.; Kim, S.-C.; Han, S. S. Enhanced Physical, Mechanical, and Cytocompatibility Behavior of Polyelectrolyte Complex Hydrogels by Reinforcing Halloysite Nanotubes and Graphene Oxide. Compos. Sci. Technol. 2019, 175, 35-45.
(32) Niu, D.; Li, Y.; Shi, J. Silica/Organosilica Cross-Linked Block Copolymer Micelles: A Versatile Theranostic Platform. Chem. Soc. Rev. 2017, 46, 569-585.
(33) Rao, J.; McClements, D. J. Formation of Flavor Oil Microemulsions, Nanoemulsions and Emulsions: Influence of Composition and Preparation Method. J. Agric. Food Chem. 2011, 59, 5026-5035.
(34) Rao, J.; McClements, D. J. Formation of Flavor Oil Microemulsions, Nanoemulsions and Emulsions: Influence of Composition and Preparation Method. J. Agric. Food Chem. 2011, 59, 5026-5035.
(35) Bugaj, L. J.: Choksi, A. T.; Mesuda, C. K.: Kane, R. S.; Schaffer, D. V. Optogenetic Protein Clustering and Signaling Activation in Mammalian Cells. Nat. Methods 2013, 10, 249-252.
1. Burdick, J. A. & Murphy, W. L. Moving from static to dynamic complexity in hydrogel design. Nat. Commun. 3, (2012).
2. Ruskowitz, E. R. & Deforest, C. A. Photoresponsive biomaterials for targeted drug delivery and 4D cell culture. Nature Reviews Materials 3, (2018).
3. Shadish, J. A., Benuska, G. M. & DeForest, C. A. Bioactive site-specifically modified proteins for 4D patterning of gel biomaterials. Nat. Mater. 18, (2019).
4. Niu, J. et al. Engineering live cell surfaces with functional polymers via cytocompatible controlled radical polymerization. Nat. Chem. 9, 537-545 (2017).
5. Repina, N. A. et al. Optogenetic control of Wnt signaling for modeling early embryogenic patterning with human pluripotent stem cells. bioRxiv 665695 (2019). doi:10.1101/665695
6. Repina, N. A., Rosenbloom, A., Mukherjee, A., Schaffer, D. V. & Kane, R. S. At Light Speed: Advances in Optogenetic Systems for Regulating Cell Signaling and Behavior. Annu. Rev. Chem. Biomol. Eng. 8, 13-39 (2017).
7. Hu, W. et al. Optogenetics sheds new light on tissue engineering and regenerative medicine. Biomaterials 227, (2020).
8. Rapp, T. L. & DeForest, C. A. Visible Light-Responsive Dynamic Biomaterials: Going Deeper and Triggering More. Advanced Healthcare Materials 9, 1901553 (2020).
9. Urciuolo, A. et al. Intravital three-dimensional bioprinting. Nat. Biomed. Eng. 4, 901-915 (2020).
10. Lee, S. H., Moon, J. J. & West, J. L. Three-dimensional micropatterning of bioactive hydrogels via two-photon laser scanning photolithography for guided 3D cell migration. Biomaterials 29, 2962-2968 (2008).
11. All, A. H. et al. Expanding the Toolbox of Upconversion Nanoparticles for In Vivo Optogenetics and Neuromodulation. Adv. Mater. 1803474 (2019). doi:10.1002/adma.201803474
12. Chen, S. et al. Near-infrared deep brain stimulation via upconversion nanoparticle-mediated optogenetics. Science (80-.). 359, 679-484 (2018).
13. Kimizuka, N. et al. Near-infrared optogenetic genome engineering based on photon upconversion hydrogels. Angew. Chemie Int. Ed. (2019). doi:10.1002/anie.201911025
14. Gargas, D. J. et al. Engineering bright sub-10-nm upconverting nanocrystals for single-molecule imaging. Nat. Nanotechnol. 9, 300-305 (2014).
15. Pun, A. B., Campos, L. M. & Congreve, D. N. Tunable Emission from Triplet Fusion Upconversion in Diketopyrrolopyrroles. J. Am. Chem. Soc. 141, 3777-3781 (2019).
16. Ravetz, B. D. et al. Photoredox catalysis using infrared light via triplet fusion upconversion. Nature 565, 343-346 (2019).
17. Goldschmidt, J. C. & Fischer, S. Upconversion for photovoltaics—a review of materials, devices and concepts for performance enhancement. Adv. Opt. Mater. 3, 510-535 (2015).
18. Ash, C., Dubec, M., Donne, K. & Bashford, T. Effect of wavelength and beam width on penetration in light-tissue interaction using computational methods. Lasers Med. Sci. 32, 1909-1918 (2017).
19. Stolik, S., Delgado, J. A., Pérez, A. & Anasagasti, L. Measurement of the penetration depths of red and near infrared light in human “ex vivo” tissues. J. Photochem. Photobiol. B Biol. 57, 90-93 (2000).
20. Hu, W. et al. Optogenetics sheds new light on tissue engineering and regenerative medicine. Biomaterials 227, (2020).
21. Bharmoria, P. et al. Simple and Versatile Platform for Air-Tolerant Photon Upconverting Hydrogels by Biopolymer-Surfactant-Chromophore Co-assembly. J. Am. Chem. Soc. 140, 10848-10855 (2018).
22. Askes, S. H. C. & Bonnet. S. Solving the oxygen sensitivity of sensitized photon upconversion in life science applications. Nature Reviews Chemistry 2, 437-452 (2018).
23. Bharmoria, P. et al. Simple and Versatile Platform for Air-Tolerant Photon Upconverting Hydrogels by Biopolymer-Surfactant-Chromophore Co-assembly. J. Am. Chem. Soc. 140, 10848-10855 (2018).
24. Huang, L. et al. Designing next generation of photon upconversion: Recent advances in organic triplet-triplet annihilation upconversion nanoparticles. Biomaterials 201, 77-86(2019).
25. Taslimi, A. et al. An optimized optogenetic clustering tool for probing protein interaction and function. Nat. Commun. 5, 1-9 (2014).
26. Bharmoria, P., Bildirir, H. & Moth-Poulsen, K. Triplet-triplet annihilation based near infrared to visible molecular photon upconversion. Chem. Soc. Rev. (2020). doi:10.1039/D0CS00257G.
27. Bharmoria, P., Yanai, N. & Kimizuka, N. Recent progress in photon upconverting gels. Gels 5, 1-10 (2019).
28. Sanders, S. N., Gangishetty, M. K., Sfeir, M. Y. & Congreve, D. N. Photon Upconversion in Aqueous Nanodroplets. J. Am. Chem. Soc. 141, 9180-9184 (2019).
29. Li, X. et al. Arabidopsis cryptochrome 2 (CRY2) functions by the photoactivation mechanism distinct from the tryptophan (trp) triad-dependent photoreduction. Proc. Natl. Acad. Sci. U.S.A. 103, 20844-20849 (2011).
30. Nishimura, N. et al. Photon Upconversion from Near-Infrared to Blue Light with TIPS-Anthracene as an Efficient Triplet-Triplet Annihilator. ACS Mater. Lett. 1, 660-664 (2019).
31. Nishimura, N. et al. Photon Upconversion from Near-Infrared to Blue Light with TIPS-Anthracene as an Efficient Triplet-Triplet Annihilator. ACS Mater. Lett. 1, 660-664(2019).
32. Fallon, K. J. et al. Molecular Engineering of Chromophores to Enable Triplet-Triplet Annihilation Upconversion. J. Am. Chem. Soc. jacs.0c06386 (2020). doi:10.1021/jacs.0c06386
33. Liu, Q. et al. Highly Photostable Near-IR-Excitation Upconversion Nanocapsules Based on Triplet-Triplet Annihilation for in Vivo Bioimaging Application. ACS Appl. Mater. Interfaces 10, 9883-9888 (2018).
34. Ma, J. et al. A green solvent for operating highly efficient low-power photon upconversion in air. Phys. Chem. Chem. Phys. 21, (2019).
35. Li, Y., Meng, H., Liu, Y. & Lee, B. P. Fibrin gel as an injectable biodegradable scaffold and cell carrier for tissue engineering. Scientific World Journal 2015, 1-10 (2015).
36. Keung, A. J., Kumar, S. & Schaffer, D. V. Presentation counts: Microenvironmental regulation of stem cells by biophysical and material cues. Annu. Rev. Cell Dev. Biol. 26, 533-356(2010).
37. Little, L., Healy, K. E, & Schaffer, D, Engineering biomaterials for synthetic neural stem cell microenvironments. Chem. Rev. 108, 1787-1796 (2008).
38. Higuchi, A., Ling, Q. D., Chang, Y., Hsu, S. T. & Umezawa, A. Physical cues of biomaterials guide stem cell differentiation fate. Chemical Reviews 113, 3297-3328 (2013).
39. Tran, H. et al. Hierarchically ordered nanopatterns for spatial control of biomolecules. ACS Nano 8, 11846-11853 (2014).
40. Chen, C. S., Mrksich, M., Huang, S., Whitesides, G. M. & Ingber, D. E. Geometric control of cell life and death. Science (80-.). 276, 1425-1428 (1997).
41. Kilian, K. A., Bugarija, B., Lahn, B. T. & Mrksich, M. Geometric cues for directing the differentiation of mesenchymal stem cells. Proc. Natl. Acad. Sci. U.S.A. 107, 4872-4877 (2010).
42. Wan, L. Q. et al. Micropatterned mammalian cells exhibit phenotype-specific left-right asymmetry. Proc. Natl. Acad. Sci. U.S.A. 108, 12295-12300 (2011).
43. Kloxin, A. M., Kasko, A. M., Salinas, C. N. & Anseth, K. S. Photodegradable hydrogels for dynamic tuning of physical and chemical properties. Science (80-.). 324, 59-43 (2009).
44. Sommer, J. R.; Shelton, A. H.; Parthasarathy, A.; Ghiviriga, I.; Reynolds, J. R.; Schanze, K. S. Photophysical Properties of Near-Infrared Phosphorescent x-Extended Platinum Porphyrins. Chem. Mater. 2011, 23, 5296-5304.
45. Yusa, K.; Rad, R.; Takeda, J.; Bradley, A. Generation of Transgene-Free Induced Pluripotent Mouse Stem Cells by the PiggyBac Transposon. Nat. Methods 2009, 6, 363-369.
46. Fallon, K. J.; Churchill, E. M.: Sanders, S. N.; Shee, J.; Weber, J. L.: Meir, R.; Reichman, D. R.; Sfeir, M. Y.; Congreve, D. N.; Campos, L. M. Molecular Engineering of Chromophores to Enable Triplet-Triplet Annihilation Upconversion. J. Am. Chem. Soc. 2020. https://pubs.acs.org/doi/10.1021/jacs.0c06386
47. Nicole A. Repina, Xiaoping Bao, Joshua A. Zimmermann, David A. Joy, Ravi S. Kane, David V. Schaffer, Optogenetic control of Wnt signaling for modeling early embryogenic patterning with human pluripotent stem cells, bioRxiv preprint doi: https://doi.org/10.1101/665695; this version posted Jun. 10, 2019.
48. Yoichi Sasaki, Mio Oshikawa, Pankaj Bharmoria, Hironori Kouno, Akiko Hayashi-Takagi, Moritoshi Sato, Itsuki Ajioka, Nobuhiro Yanai, and Nobuo Kimizuka, Near-infrared Optogenetic Genome Engineering Based on Photon-Upconversion Hydrogels, Angew. Chem. Int. Ed. 2019, 58, 17827-17833, https://onlinelibrary.wiley.com/doi/abs/10.1002/anie.201911025.
49. S. G. Yüz, S. Rasoulinejad, M. Mueller, A. E. Wegner, S. V. Wegner, Adv. Biosyst. 2019, 3, 1800310.
50. F. G. Downs, D. J. Lunn, M. J. Booth, J. B. Sauer, W. J. Ramsay, R, G. Klemperer, C. J. Hawker, H. Bayley, Nat. Chem. 2020, 12, 363.
51. N. Gupta, B. F. Lin, L. M. Campos, M. D. Dimitriou, S. T. Hikita, N. D. Treat, M. V. Tirrell, D. O. Clegg, E. J. Kramer, C. J. Hawker, Nat. Chem. 2010, 2, 138.
52. A. M. Rosales, K. S. Anseth, Nat. Rev. Mater. 2016, 1, 1.
53. C. A. DeForest, K. S. Anseth, Nat. Chem. 2011, 3, 925.
54. B. Maiti, A. Abramov, R. Pérez-Ruiz, D. Diaz Diaz, Acc. Chem. Rev. 2019, 52, 1865.

1. A biomaterial, comprising:
a biocompatible scaffold,
a cell, and
an emulsion droplet containing triplet fusion upconversion chromophores that emit a visible light upon an excitation with a near-infrared light,
wherein the cell and the emulsion droplet are associated with the biocompatible scaffold.

2. The biomaterial of claim 1, wherein the biocompatible scaffold comprises at least one of (i) a hydrogel, or (ii) a fibrinogen hydrogel.

3. (canceled)

4. The biomaterial of claim 1, wherein the cell includes an engineered multipotent stem cell, an engineered pluripotent stem cell, a fibroblast cell, an endothelial cell, or a combination thereof.

5. The biomaterial of claim 1, wherein the cell expresses a light activated protein.

6. The biomaterial of claim 5, wherein the light activated protein is cryptochrome 2.

7. The biomaterial of claim 1, wherein the triplet fusion upconversion chromophores include a sensitizer chromophore and an annihilator chromophore.

8. The biomaterial of claim 1, wherein the biomaterial is at least one of a 3D material or non-cytotoxic.

9. (canceled)

10. The biomaterial of claim 1, wherein the triplet fusion upconversion chromophores comprise at least one selected from the group consisting of diketopyrrolopyrrole, anthracene, tetracene, and derivatives thereof.

11. The biomaterial of claim 1, wherein the emulsion droplet contains at least one of (i) a surfactant and the surfactant that comprises Pluronic F127, or (ii) a natural oil.

12. (canceled)

13. The biomaterial of claim 1, further comprising a layer containing silica disposed on at least a portion of a surface of the emulsion droplet.

14. The biomaterial of claim 1, wherein the near-infrared light has a wavelength in a range of from about 700 nm to about 1400 nm.

15. The biomaterial of claim 14, wherein the range of the wavelength of the near-infrared light is from about 700 nm to about 750 nm, from about 750 nm to about 800 nm, from about 800 nm to about 850 nm, from about 850 nm to about 1000 nm, or from about 1000 nm to about 1400 nm

16. The biomaterial of claim 1, wherein the triplet fusion upconversion chromophores upon the excitation with the near-infrared light emit light having a wavelength in a range of about 390 nm to about 550 nm, or about 390 nm to about 450 nm, or about 400 nm to about 500 nm, or about 450 nm to about 550 nm.

17. A micelle, comprising triplet fusion upconversion chromophores that emit a visible light upon excitation with a near-infrared light, a surfactant and a natural oil.

18. The micelle of claim 17, wherein the triplet fusion upconversion chromophores include a sensitizer chromophore and an annihilator chromophore.

19. The micelle of claim 17, wherein the triplet fusion upconversion chromophores comprise at least one selected from the group consisting of diketopyrrolopyrrole, anthracene, tetracene, and derivatives thereof.

20. The micelle of claim 17, wherein the surfactant comprises Pluronic F127.

21. The micelle of claim 17, wherein the near-infrared light has wavelength in a range of from about 700 nm to about 1400 nm, for example, 700 nm to about 750 nm, from about 750 nm to about 800 nm, from about 800 nm to about 850 nm, from about 850 nm to about 1000 nm, or from about 1000 nm to about 1400 nm.

22. The micelle of claim 17, wherein the triplet fusion upconversion chromophores upon the excitation with the near-infrared light emit light having a wavelength in a range of about 390 nm to about 550 nm, or about 390 nm to about 450 nm, or about 400 nm to about 500 nm, or about 450 nm to about 550 nm.

23. A nanoparticle, comprising:
a micelle comprising triplet fusion upconversion chromophores that emit a visible light upon excitation with a near-infrared light, a surfactant and a natural oil; and
a layer containing silica disposed on at least a portion of a surface of the micelle.

24. The nanoparticle of claim 23, wherein the triplet fusion upconversion chromophores include a sensitizer chromophore and an annihilator chromophore.

25. The nanoparticle of claim 23, wherein the triplet fusion upconversion chromophores comprise at least one selected from the group consisting of diketopyrrolopyrrole, anthracene, tetracene, and derivatives thereof.

26. The nanoparticle of claim 23, wherein the surfactant comprises Pluronic F127.

27. The nanoparticle of claim 23, wherein the near-infrared light has wavelength in a range of from about 700 nm to about 1400 nm, for example, 700 nm to about 750 nm, from about 750 nm to about 800 nm, from about 800 nm to about 850 nm, from about 850 nm to about 1000 nm, or from about 1000 nm to about 1400 nm.

28. The nanoparticle of claim 23, wherein the triplet fusion upconversion chromophores upon the excitation with the near-infrared light emit light have a wavelength in a range of about 390 nm to about 550 nm, or about 390 nm to about 450 nm, or about 400 nm to about 500 nm, or about 450 nm to about 550 nm.
